(19) United States
(12) Patent Application Publication
(10) Pub. No.: US 2022/0177979 A1
(43) Pub. Date: Jun. 9, 2022

(54) CLINICAL PROGNOSTICATION TEST IN UVEAL MELANOMA

(71) Applicants: Samuel Bakhoum, La Jolla, CA (US); Ashley Laughney, La Jolla, CA (US); The Regents of the University of California, Oakland, CA (US)

(72) Inventors: Mathieu Bakhoum, La Jolla, CA (US); Samuel Bakhoum, La Jolla, CA (US); Paul Mischel, La Jolla, CA (US); Ashley Laughney, La Jolla, CA (US)

(21) Appl. No.: 17/601,897

(22) PCT Filed: Apr. 8, 2020
(86) PCT No.: PCT/US2020/027225

§ 371 Date: Oct. 6, 2021

Related U.S. Application Data
(60) Provisional application No. 62/831,552, filed on Apr. 9, 2019.

(51) Int. Cl. C12Q 1/6886

(52) U.S. Cl. CPC C12Q 1/6886; C12Q 2600/158; C12Q 2600/154; C12Q 2600/106

(57) ABSTRACT

Provided herein are methods for treating uveal melanoma in a subject in need thereof, and methods of detecting high-risk uveal melanoma in a subject, the subject having been selected by a method comprising, or alternatively consisting essentially of, or yet further consisting of detection of high methyl ati on levels at one or more of: BAP-1, CCNG2 and/or FKBP14 and/or measuring the mRNA expression of JARID2 and TMEM173 and determining ratio of mRNA expression JARID2 to TMEM173 in a sample isolated from the subject. Further provided herein are methods of determining prognosis of a subject having uveal melanoma, wherein high methylation levels and/or the ratio of mRNA expression JARID2 to TMEM173 of less than about 1 indicate a decreased probability and/or duration of survival and low methylation levels and/or the ratio of mRNA expression JARID2 to TMEM173 of greater than about 1 indicate an increased probability and/or duration of survival.

### CROSS-REFERENCE TO RELATED APPLICATIONS

This application claims priority under 35 U.S.C. § 119(e) to U.S. Provisional Application No. 62/831,552, filed Apr. 9, 2019, the contents of which is hereby incorporated by reference in its entirety.

### BACKGROUND

The following discussion of the background is merely provided to aid the reader in the understanding the disclosure and is not admitted to describe or constitute prior art to the present disclosure. Uveal melanoma commonly known as ocular or choroidal melanoma, is a rare cancer of the eye. Uveal melanoma (UM) is the most common primary intraocular cancer and second type of melanomas. Ocular melanoma is diagnosed in approximately 2,000-2,500 adults annually in the United States. In both the U.S. and Europe, this equates to about 5-7.5 cases per million people per year and, for people over 50 years old, the incidence rate increases to around 21 per million per year. While the primary tumor is highly treatable, about half of the patients will develop metastasis—typically to the liver. Metastatic disease is universally fatal. Once metastases are detected, median survival is less than six months. Patients rarely survive beyond two years. While traditional staging methods such as tumor size and location, still play a role in assessing metastatic risk, they are rarely used to individualize patient management plans.

### SUMMARY

This disclosure also provides methods of determining one or more of the following: (1) prognosis of a subject having uveal melanoma (UM), (2) an probability of having a high-risk UM with poor prognosis, (3) a probability of having a UM tumor with partial or full monosomy 3, or (4) a probability of having a BAP1 mutation in a UM. The method comprises, or alternatively consists essentially of, or yet further consists of detecting methylation levels at one or more of: BAP-1, CCNG2 and/or FKBP14 and/or determining ratio of mRNA expression JARID2 to TMEM173 in a sample isolated from the subject. High methylation levels and/or a low ratio of mRNA expression JARID2 to TMEM173 indicate a less favorable prognosis and low methylation levels and/or a high ratio of mRNA expression JARID2 to TMEM173 indicate a more favorable prognosis. In certain aspects, an intermediate methylation level and/or an intermediate ratio of mRNA expression JARID2 to TMEM173 indicate an intermediate prognosis which comprises an intermediate overall survival, and/or an intermediate probability of having a metastasis. As used herein, a less favorable prognosis comprises, or alternatively consists essentially of, or yet further consists of a decreased overall survival, and/or an increased probability of having a metastasis (for example in liver) while more favorable prognosis comprises, or alternatively consists essentially of, or yet further consists of an increased overall survival and/or a decreased probability of having a metastasis (such as in liver). An overall survival may refer to probability and/or duration of survival. In a further aspect, the method (for example, the determination step of the method) comprises, or alternatively consists essentially of, or yet further consists of measuring the mRNA expression of JARID2 and TMEM173 and determining a ratio of mRNA expression JARID2 to TMEM173 in a sample isolated from the subject. In one aspect, the method further comprises, or alternatively consists essentially of, or yet further consists of detecting methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, wherein high methylation levels indicate a less favorable prognosis and low methylation levels indicate more favorable prognosis. In another aspect, the method (for example, the method step of detection and/or determination) comprises, or alternatively consists essentially of, or yet further consists of detecting methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or determining ratio of mRNA expression JARID2 to TMEM173 in a sample isolated from the subject. The high methylation levels and/or the low ratio of mRNA expression JARID2 to TMEM173 indicate a less favorable prognosis and the low methylation levels and/or the high ratio of mRNA expression JARID2 to TMEM173 indicate more favorable prognosis. In a further aspect, the method (for example, the detection and/or determination step of the method) comprises, or alternatively consists essentially of, or yet further consists of measuring the mRNA expression of JARID2 and TMEM173 and determining a ratio of mRNA expression JARID2 to TMEM173 in a sample isolated from the subject. In yet a further aspect, the method further comprises, or alternatively consists essentially of, or yet further consists of administering to the subject an effective amount of one or more of: an anti-cancer drug, immunotherapy, chemotherapy, and/or radiation therapy suitable for the determined prognosis and/or probability of a subject, thereby treating the subject. The detection and/or measurement can be performed in vitro and/or ex vivo.

This disclosure provides methods for treating uveal melanoma and in one aspect high risk uveal melanoma in a subject selected for the treatment, the method comprising, or alternatively consisting essentially of, or yet further consisting of administering to the subject an effective amount of one or more of: an anti-cancer drug, immunotherapy, chemotherapy, and/or radiation therapy selective for the uveal melanoma (for example, the high-risk uveal melanoma), thereby treating the subject. In one aspect, the subject is selected for the therapy by assaying and detecting in a sample isolated from the subject for one or more of the following: a high methylation level at one or more of: BAP-1, CCNG2 and/or FKBP14; a low mRNA expression ratio of JARID2 to TMEM173; a high methylation level at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2; and/or a high methylation level at one or more of the methylation loci identified in Table 2 and/or 7.

In another aspect, the subject is selected for the therapy by detecting in the sample isolated from the subject high methylation levels at one or more of: BAP-1, CCNG2, FKBP14, PDE4B, SH3D19, SLC6A15 LMCD1, LMCD1-AS1, SENP6, NFIA, CTD-2207023.12, ZNF358, CCDC176, ENTPD5, DTWD1, FAM227B, SESN1, ENPP2 and/or methylation loci identified in Table 2 and/or 7, and/or determining a low ratio of mRNA expression JARID2 to TMEM173.

In a further aspect, the subject is selected for the therapy by measuring the mRNA expression of JARID2 and TMEM173 and determining a low ratio of mRNA expression JARID2 to TMEM173 in a sample isolated from the subject. In one aspect, the low ratio of mRNA expression JARID2 to TMEM173 is less than about 1, less than about 2 or less than about 0.3.

Also provided is a method for treating a low-risk uveal melanoma in a subject selected for the treatment by a method comprising, or alternatively consisting essentially of, or yet further consisting of administering an effective amount of one or more of: an anti-cancer drug, immunotherapy, chemotherapy, and/or radiation therapy selective for the uveal melanoma (for example, the low-risk uveal melanoma), thereby treating the subject. The subject is selected for the therapy when the sample isolated from the subject has one or more of the following: a low methylation levels at one or more of: BAP-1, CCNG2 and/or FKBP14; a high mRNA expression ratio of JARID2 to TMEM173; a low methylation level at one or more of: PDE4B, SH3D19, SLC6A15 LMCD1, LMCD1-AS1, SENP6, NFIA, CTD-2207023.12, ZNF358, CCDC176, ENTPD5, DTWD1, FAM227B, SESN1, ENPP2; and/or a low methylation level at one or more of the methylation loci identified in Table 2 and/or 7.

In a further aspect, the subject is selected for the treatment by a method that comprise, or alternatively consist essentially of, or yet further consist of measuring the mRNA expression of JARID2 and TMEM173 and determining a high ratio of mRNA expression JARID2 to TMEM173 in a sample isolated from the subject. In yet a further aspect, the high ratio of mRNA expression JARID2 to TMEM173 is (a) about 1 and/or greater than about 1, (b) about 2 and/or greater than about 2, or (c) about 0.3 and/or greater than about 0.3.

Additionally provided is a method for treating an intermediate-risk uveal melanoma in a subject selected for the therapy comprising, or alternatively consisting essentially of, or yet further consisting of administering an effective amount of one or more of: an anti-cancer drug, immunotherapy, chemotherapy, and/or radiation therapy selective for the uveal melanoma (for example, the intermediate-risk uveal melanoma), thereby treating the subject. In one aspect, the subject is selected for the method by detecting in a sample isolated from the subject one or more of the following an intermediate methylation levels at one or more of: BAP-1, CCNG2 and/or FKBP14; an intermediate mRNA expression ratio of JARID2 to TMEM173; an intermediate methylation level at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2; and/or an intermediate methylation level at one or more of the methylation loci identified in Table 2 and/or 7.

In a further aspect, the subject is selected for the method by detecting in a sample mRNA expression of JARID2 and TMEM173 and determining an intermediate ratio of mRNA expression JARID2 to TMEM173 in the sample isolated from the subject. In yet a further aspect, the intermediate ratio of mRNA expression JARID2 to TMEM173 is about 0.3 to about 1, from about 0.3 to about 2, or from about 1 to about 2.

Further provided herein is a method for detecting high-risk uveal melanoma in a subject comprising, or alternatively consisting essentially of, or yet further consisting of detecting high methylation levels at one or more of: BAP-1, CCNG2 and/or FKBP14 and/or a low ratio of mRNA expression JARID2 to TMEM173 in a sample isolated from the subject. In a further aspect, the method (for example, the detection step of the method) comprises, or alternatively consists essentially of, or yet further consists of measuring the mRNA expression of JARID2 and TMEM173 and determining a low ratio of mRNA expression JARID2 to TMEM173 in a sample isolated from the subject. In one aspect, the method further comprises, or alternatively consists essentially of, or yet further consists of detecting high methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7. In another aspect, the method of detection comprises, or alternatively consists essentially of, or yet further consists of detecting high methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or a low ratio of mRNA expression JARID2 to TMEM173 in a sample isolated from the subject. In a further aspect, the subject is selected when the ratio of mRNA expression JARID2 to TMEM173 is less than about 1, less than about 2 or less than about 0.3. Additionally or alternatively, the detection is an in vitro and/or ex vivo detection. In a further embodiment, the method further comprises, or alternatively consists essentially of, or yet further consists of administering to the subject with a high-risk uveal melanoma an effective amount of one or more of: an anti-cancer drug, immunotherapy, chemotherapy, and/or radiation therapy selective for the uveal melanoma (for example, the high-risk melanoma), thereby treating the subject.

Additionally provided herein is a method for detecting low-risk uveal melanoma in a subject comprising, or alternatively consisting essentially of, or yet further consisting of detecting low methylation levels at one or more of: BAP-1, CCNG2 and/or FKBP14 and/or a high ratio of mRNA expression JARID2 to TMEM173 in a sample isolated from the subject. In a further aspect, the method (for example, the detection step of the method) comprises, or alternatively consists essentially of, or yet further consists of measuring the mRNA expression of JARID2 and TMEM173 and determining a high ratio of mRNA expression JARID2 to TMEM173 in a sample isolated from the subject. In one aspect, the method further comprises, or alternatively consists essentially of, or yet further consists of detecting low methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7. In another aspect, the method of detection comprises, or alternatively consists essentially of, or yet further consists of detecting low methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or a high ratio of mRNA expression JARID2 to TMEM173 in a sample isolated from the subject. In a further aspect, the subject is selected when the ratio of mRNA expression JARID2 to TMEM173 is (a) about 1 and/or greater than about 1, (b) about 2 and/or greater than about 2, or (c) about 0.3 and/or less than about 0.3. Additionally or alternatively, the detection is an in vitro and/or ex vivo detection. In a further embodiment, the method further comprises, or alternatively consists essentially of, or yet further consists of administering to the subject with a low-risk uveal melanoma an effective amount of one or more of: an anti-cancer drug, immunotherapy, chemotherapy, and/or radiation therapy selective for the uveal melanoma (for example, the low-risk melanoma), thereby treating the subject.

Additionally provided herein is a method for detecting an intermediate-risk uveal melanoma in a subject comprising, or alternatively consisting essentially of, or yet further consisting of detecting intermediate methylation levels at one or more of: BAP-1, CCNG2 and/or FKBP14 and/or an intermediate ratio of mRNA expression JARID2 to TMEM173 in a sample isolated from the subject. In a further aspect, the method (for example, the detection step of the method) comprises, or alternatively consists essentially of, or yet further consists of measuring the mRNA expression of JARID2 and TMEM173 and determining an intermediate ratio of mRNA expression JARID2 to TMEM173 in a sample isolated from the subject. In one aspect, the method further comprises, or alternatively consists essentially of, or yet further consists of detecting intermediate methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7. In another aspect, the method of detection comprises, or alternatively consists essentially of, or yet further consists of detecting intermediate methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or an intermediate ratio of mRNA expression JARID2 to TMEM173 in a sample isolated from the subject. In a further aspect, the subject is selected when the ratio of mRNA expression JARID2 to TMEM173 is from about 0.3 to about 1, from about 0.3 to about 2, or from about 1 to about 2. Additionally or alternatively, the detection is an in vitro and/or ex vivo detection. In a further embodiment, the method further comprises, or alternatively consists essentially of, or yet further consists of administering to the subject with an intermediate-risk uveal melanoma an effective amount of one or more of: an anti-cancer drug, immunotherapy, chemotherapy, and/or radiation therapy selective for the uveal melanoma (for example, the intermediate-risk melanoma), thereby treating the subject.

In certain aspects of any methods as disclosed herein, a low ratio of mRNA expression JARID2 to TMEM173 is less than about 1, less than about 2 or less than about 0.3. A high ratio of mRNA expression JARID2 to TMEM173 is (a) about 1 and/or greater than about 1, (b) about 2 and/or greater than about 2, or (c) about 0.3 and/or less than about 0.3. In one aspect, an intermediate ratio of mRNA expression JARID2 to TMEM173 is from about 0.3 to about 1, from about 0.3 to about 2, or from about 1 to about 2. In an alternative aspect, a high ratio further comprises, or alternatively consists essentially of, or yet further consists of from about 0.3 to about 1, from about 0.3 to about 2, or from about 1 to about 2.

In certain aspects of any methods as disclosed herein, high methylation levels are detected as (i) a methylation beta value of (a) above about 0.3, (b) above about 0.25, (c) above about 0.75, or (d) above the cutoff for beta value set in Table 2 and/or 7; and/or (ii) an average of all methylation beta values at (a) above about 0.3, (b) above about 0.25, or (c) above about 0.75. Low methylation levels are detected as (i) a methylation beta value of (a) about 0.3 or lower, or (b) about 0.25 or lower, (c) about 0.75 or lower, or (d) the cutoff for beta value set in Table 2 and/or 7 or lower; and/or (ii) as an average of all methylation beta values at (a) about 0.3 or lower, or (b) about 0.25 or lower, or (c) about 0.75 or lower. In one aspect, the intermediate methylation levels are detected as (i) a methylation beta value from about 0.25 to about 0.75; and/or (ii) an average of all methylation beta values from about 0.25 to about 0.75. Alternatively, a low methylation level further comprises, or alternatively consists essentially of, or yet further consists of a level detected as (i) a methylation beta value from about 0.25 to about 0.75; and/or (ii) an average of all methylation beta values from about 0.25 to about 0.75.

Further provided is a method comprising quantifying a methylation level of a loci in a sample isolated from a subject. The loci comprises one or more of the loci identified in Table 2 and/or 7 and/or the loci within one or more of the following genes: BAP-1, CCNG2 and/or FKBP14, PDE4B, SH3D19, SLC6A15 LMCD1, LMCD1-AS1, SENP6, NFIA, CTD-2207023.12, ZNF358, CCDC176, ENTPD5, DTWD1, FAM227B, SESN1, or ENPP2. Optionally, a DNA comprising the one or more loci have been enriched and/or amplified prior to or concurrently with the quantification step. In one aspect, the method further comprises obtaining a sample detected with a methylation level at the one or more loci, and/or an average methylation level at more than one loci, as having a beta value of (a) about 0.3 or lower, (b) greater than about 0.3, (c) about 0.25 or lower, (d) about 0.25 to about 0.75, (e) greater than about 0.25, (f) about 0.75 or lower, (g) greater than about 0.75, (h) about the cutoff for the beta value set in Table 2 and/or 7 or lower, or (i) greater than the cutoff for the beta value set in Table 2 and/or 7. Additionally or alternatively, the method further comprises quantifying one or more of the following in the isolated sample: (a) mRNA level of JARID2 and mRNA level of TMEM173; (b) ratio of mRNA expression JARID2 to TMEM173; or (c) ratio of mRNA expression TMEM173 to JARID2.

Additionally provided is a method comprising quantifying one or more of the following in a sample isolated from a subject: (a) mRNA level of JARID2 and mRNA level of TMEM173; (b) ratio of mRNA expression JARID2 to TMEM173; or (c) ratio of mRNA expression TMEM173 to JARID2. Optionally mRNAs of JARID2 and TMEM173 have been enriched and/or reverse-transcribed prior to or concurrently with the quantification. In one aspect, the method further comprises obtaining mRNAs of JARID2 and TMEM173 having a ratio of mRNA expression JARID2 to TMEM173 at (a) less than about 0.3, (b) about 0.3 to about 1, (c) about 0.3 to about 2, (d) more than 0.3, (e) less than about 1, (f) about 1 to about 2, (g) more than about 1, (h) less than about 2, or (i) more than about 2.

In one embodiment, the BAP-1 methylation levels in the methods described herein are detected at the methylation loci identified in Tables 2, 3, 4 and/or 5. In one particular embodiment, BAP-1 methylation levels in the methods described herein are detected at the methylation locus CGI:chr3:52409661-52410118.

In one aspect, high methylation levels comprise, or alternatively consist essentially of, or yet further consist of a methylation beta value above about 0.3. In another aspect, low methylation levels comprise, or alternatively consist essentially of, or yet further consist of a methylation beta value of about 0.3 or lower.

The sample isolated from the subject is one or more of a blood, a urine, an aqueous vitreous humor, a tumor biopsy and/or a liquid biopsy sample. Biopsy specimens from fine needle aspiration or enucleation, paraffin-embedded or frozen specimens, as well as cell-free DNA from patients with uveal melanoma can also be used as samples. In a further aspect, the sample is one or more of an aqueous humor, a vitreous humor, a sample from primary or metastatic UM, a tumor tissue in freshly obtained specimens, a tumor tissue in paraffin embedded tissue, a frozen tumor tissue, a tumor tissue in fixative, cell free DAN and/or circulating tumor DNA in biological fluids. In one aspect, the subject is a mammal. In another aspect, the subject is a human. In certain aspects of the methods as disclosed herein, the subject is suspect of having a primary or metastatic uveal melanoma (UM) and/or is diagnosed with a primary or metastatic uveal melanoma. In a further aspect, the detection, measurement, determination, and/or quantification is performed in vitro and/or ex vivo.

Also described herein are kits comprising, or alternatively consisting essentially of, or yet further consisting of probes, primers, optional bisulfite salt or solution, and optional antibodies for carrying out the methods of this disclosure, and optional instructions for use. In a further aspect, the instruction for use provide directions to conduct any of the methods disclosed herein.

### BRIEF DESCRIPTION OF THE DRAWING

FIGS. 1A to 1M show that methylation at the selected loci vary differently between specimens, ranging from less than 10% to more than 100%. It also indicates where a cut-off between high and low methylation is determined for each locus. Cases were arranged in an ascending order of methylation (beta value) of gene shown on top and plotted on the x-axis, while beta-value of gene of interest (shown on top) were plotted on the y-axis. The following also provides the gene of interest for each figure panel along with the corresponding beta value cutoff as summarized in Table 2: FIG. 1A, 309887, beta value cutoff=0.30; FIG. 1B, 338430, beta value cutoff=0.20; FIG. 1C, 343359, beta value cutoff=0.50; FIG. 1D, 161744, beta value cutoff=0.50; FIG. 1E, 75967, beta value cutoff=0.70; FIG. 1F, 382471, beta value cutoff=0.40; FIG. 1G, 89586, beta value cutoff=0.30; FIG. 1H, 406409, beta value cutoff=0.40; FIG. 1I, 111951, beta value cutoff=0.50; FIG. 1J, 41573, beta value cutoff=0.50; FIG. 1K, 130560, beta value cutoff=0.35; FIG. 1L, 7446, beta value cutoff=0.60; and FIG. 1M, 267855, beta value cutoff=0.50.

FIGS. 2A to 2M show that methylation at the selected loci negatively correlates with BAP1 genomic copy number, i.e., positively correlates with BAP1 copy loss. Cases were arranged in an ascending order of methylation (beta value) of locus of interest on top and plotted on the x-axis, while relative BAP1 copy number alteration were plotted on the y-axis. The following also lists the locus of interest for each figure panel: FIG. 2A, 309887; FIG. 2B, 338430; FIG. 2C, 343359; FIG. 2D, 161744; FIG. 2E, 75967; FIG. 2F, 382471; FIG. 2G, 89586; FIG. 2H, 406409; FIG. 2I, 111951; FIG. 2J, 41573; FIG. 2K, 130560; FIG. 2L, 7446; and FIG. 2M, 267855.

FIGS. 3A to 3N show that methylation at the selected loci can predict genomic copy number of BAP1 (and/or BAP1 copy loss). Methylation cutoff defined for each locus was used while BAP1 Copy loss was defined as <−0.2. Sensitivity, specificity, positive predictive value (PPV) and negative predictive value (NPV) were calculated and noted as illustrated in FIG. 3A. The locus of interest for each figure panel is listed below: FIG. 3B, 309887, beta value cutoff=0.30; FIG. 3C, 338430, beta value cutoff=0.20; FIG. 3D, 343359, beta value cutoff=0.50; FIG. 3E, 161744, beta value cutoff=0.50; FIG. 3F, 75967, beta value cutoff=0.70; FIG. 3G, 382471, beta value cutoff=0.40; FIG. 3H, 89586, beta value cutoff=0.30; FIG. 3I, 406409, beta value cutoff=0.40; FIG. 3J, 111951, beta value cutoff=0.50; FIG. 3K, 41573, beta value cutoff=0.50; FIG. 3L, 130560, beta value cutoff=0.35; FIG. 3M, 7446, beta value cutoff=0.60; and FIG. 3N, 267855, beta value cutoff=0.50.

FIG. 4 shows correlation between BAP1 methylation and chromosome 3 status. Methylation levels of BAP1 were plotted on the x-axis while chromosome 3 copy numbers were plotted on the y-axis. Statistical analysis was performed. Pearson: −0.8642 (−0.9110 to −0.7956), P<0.0001.

FIG. 5 shows correlation between BAP1 methylation and chromosome 3 loss (partial or complete). Methylation levels of BAP1 were plotted on the x-axis while chromosome 3 copy numbers were plotted on the y-axis. Statistical analysis was performed. Pearson: −0.4233 (−0.6398 to −0.1446), P=0.0042.

FIG. 6 shows correlation between average methylation of all loci and chromosome 3 status. Average beta values (methylation) of all loci were plotted on the x-axis while chromosome 3 copy/BAP1 copy numbers were plotted on the y-axis. Statistical analysis was performed. Pearson: −0.9456 (−0.9649 to −0.9162), P<0.0001.

FIG. 7 shows correlation between average methylation of all loci and chromosome 3 loss (partial or complete). Average beta values (methylation) of all loci were plotted on the x-axis while chromosome 3 copy/BAP1 copy numbers were plotted on the y-axis. Statistical analysis was performed. Pearson: −0.6347 (−0.7839 to −0.4163), P<0.0001.

FIG. 8 provides a bar graph showing correlation between chromosome 3 copy and BAP1 methylation. P<0.0001.

FIG. 9 provides a bar graph showing correlation between chromosome 3 copy and all loci methylation. P<0.0001.

FIGS. 10A to 10N show that methylation at the selected loci can predict BAP1 mutational status. Methylation cutoff defined for each locus was used. Sensitivity, specificity, positive predictive value (PPV) and negative predictive value (NPV) were calculated and noted as illustrated in FIG. 10A. The locus of interest for each figure panel is listed below: FIG. 10B, 309887, beta value cutoff=0.30; FIG. 10C, 338430, beta value cutoff=0.20; FIG. 10D, 343359, beta value cutoff=0.50; FIG. 10E, 161744, beta value cutoff=0.50; FIG. 10F, 75967, beta value cutoff=0.70; FIG. 10G, 382471, beta value cutoff=0.40; FIG. 10H, 89586, beta value cutoff=0.30; FIG. 10I, 406409, beta value cutoff=0.40; FIG. 10J, 111951, beta value cutoff=0.50; FIG. 10K, 41573, beta value cutoff=0.50; FIG. 10L, 130560, beta value cutoff=0.35; FIG. 10M, 7446, beta value cutoff=0.60; and FIG. 10N, 267855, beta value cutoff=0.50.

FIG. 11 provides a graph showing correlation between BAP1 methylation and mutations in BAP1.

FIG. 12 provides a bar graph showing fraction of tumors with BAP1 mutations based on BAP1 methylation. In each group (beta value <0.3 or >0.3), the left bar provides fraction of tumors having wildtype BAP1 and the right bar provides fraction of tumors having BAP1 mutations. Statistical analysis was performed. Fisher's exact test shows P<0.0001. Sensitivity: 0.6379 (0.5093 to 0.7495); Specificity: 0.9545 (0.7820 to 0.9977); Positive Predictive Value: 0.9737 (0.8651 to 0.9987); Negative Predictive Value: 0.5000 (0.3553 to 0.6447), and Likelihood Ratio: 14.03.

FIG. 13 provides a graph showing correlation between average methylation of all loci and mutations in BAP1.

FIG. 14 provides a bar graph showing fraction of tumors with BAP1 mutations based on average methylation of all loci. In each group (beta value <0.3 or >0.3), the left bar provides fraction of tumors having wildtype BAP1 and the right bar provides fraction of tumors having BAP1 mutations. Statistical analysis was performed. Fisher's exact test shows P<0.0001. Sensitivity: 0.6552 (0.5267 to 0.7644); Specificity: 1.000 (0.8513 to 1.000); Positive Predictive Value: 1.000 (0.9082 to 1.000); and Negative Predictive Value: 0.5238 (0.3772 to 0.6664).

FIGS. 15A to 15N show that methylation at the selected loci can predict overall survival. Methylation cutoff defined for each locus as well as the overall survival was used. Sensitivity, specificity, positive predictive value (PPV) and negative predictive value (NPV) were calculated and noted as illustrated in FIG. 15A. The locus of interest for each figure panel is listed below: FIG. 15B, 309887, beta value cutoff=0.30; FIG. 15C, 338430, beta value cutoff=0.20; FIG. 15D, 343359, beta value cutoff=0.50; FIG. 15E, 161744, beta value cutoff=0.50; FIG. 15F, 75967, beta value cutoff=0.70; FIG. 15G, 382471, beta value cutoff=0.40; FIG. 15H, 89586, beta value cutoff=0.30; FIG. 15I, 406409, beta value cutoff=0.40; FIG. 15J, 111951, beta value cutoff=0.50; FIG. 15K, 41573, beta value cutoff=0.50; FIG. 15L, 130560, beta value cutoff=0.35; FIG. 15M, 7446, beta value cutoff=0.60; and FIG. 15N, 267855, beta value cutoff=0.50.

FIG. 16 provides survival based on methylation level of each loci. For each panel, the upper line shows survival data of patient having a methylation level of the locus of interest (shown on top of each panel) lower than the cutoff value set in Table 2, while the lower line shows survival data of patient having a methylation level above the cutoff value.

FIG. 17 provides a graph showing overall survival based on BAP1 methylation. The upper line shows survival data of patient having a BAP1 methylation level lower than 0.3, while the lower line shows survival data of patient having a BAP1 methylation level above 0.3. Hazard ratio (HR): 14.69 (6.411 to 33.64). P<0.0001.

FIG. 18 provides a graph showing overall survival based on BAP1 methylation at the locus of 309887 (Tertiles). The upper line shows survival data of the bottom third of the patients ranked based on the BAP1 methylation level (i.e., having a low methylation level compared to the rest patients, for example having a beta value less than about 0.23), and the middle line shows survival data of the center third of the patients ranked based on the BAP1 methylation level (i.e., having an intermediate methylation level compared to the rest patients, for example having a beta value from about 0.23 to about 0.53), while the lower line shows survival data of the top third of the patients ranked based on the BAP1 methylation level (i.e., having a high methylation level compared to the rest patients, for example having a beta value greater than 0.53).

FIG. 19 provides a graph showing overall survival based on average methylation of all loci. The upper line shows survival data of patient having a BAP1 methylation level lower than 0.25, and the middle line shows survival data of patient having a BAP1 methylation level between 0.25 and 0.75, while the lower line shows survival data of patient having a BAP1 methylation level above 0.75. P<0.0001.

FIG. 20 provides a graph showing overall survival based on methylation of all loci using individual biomarker cutoffs. The upper line shows survival data of patient having a methylation level lower than the cutoff for each locus, while the lower line shows survival data of patient having a methylation level higher than the cutoff for each locus. HR 16.50, (6.787 to 40.10). P<0.0001.

FIG. 21 provides a graph showing distant metastasis based on BAP1 methylation values. Percentages of patients free of metastasis were plotted on the y-axis. The upper line shows data from patients having a BAP1 methylation level lower than 0.3, while the lower line shows data from patient having a BAP1 methylation level higher than 0.3. HR 5.482 (1.656 to 18.14). P=0.0014. n=60.

FIG. 22 provides a graph showing distant metastasis based on average methylation of all loci. Percentages of patients free of metastasis were plotted on the y-axis. The upper line shows data from patients having an average methylation level lower than 0.3, while the lower line shows data from patient having an average methylation level higher than 0.3. HR 4.890 (1.518 to 15.76). P=0.0033.

FIG. 23 shows that survival at 5 years was 100% for JARID2 and TMEM173 (J/T) ratio >1 and 18% for J/T ratio <1. There were 23 patients in the first category (J/T ratio >1) and 55 samples in the second category (J/T ratio <1).

FIG. 24 provides overall survival of patients with UM stratified based on JARID2 levels relative to STING (TMEM73) levels. The upper line shows survival data of patient having a JARID2/STING ratio above 1, and the lower line shows survival data of patient having a JARID2/STING ratio below 1. HR 15.52 (6.769 to 35.59). P=0.0033.

FIG. 25 provides Kaplan Meier curves showing that stratifying patients based on JARID2/STING (TMEM173) ratios (>2, 1-2, 0.3-1 or less than 0.3) provides better prognostication for overall survival. The top line shows survival data of patient having a JARID2/STING ratio above 2, while the second line from the top shows survival data of patient having a JARID2/STING ratio from 1 to 2. The bottom line shows survival data of patient having a JARID2/STING ratio below 0.3, and the second line from the bottom shows survival data of patient having a JARID2/STING ratio from 0.3 to 1.

FIG. 26 shows fraction of tumors with BAP1 mutations based on JARID2/STING ratio. In each group (JARID2/STING ratio >1 or <1), the left bar provides fraction of tumors having wildtype BAP1 and the right bar provides fraction of tumors having BAP1 mutations.

FIG. 27 shows correlation between JARID2/STING (TMEM173) ratio and chromosome 3 copy number.

FIG. 28 provides a bar graph showing correlation between JARID2/STING (TMEM173) ratio and chromosome 3 copy number.

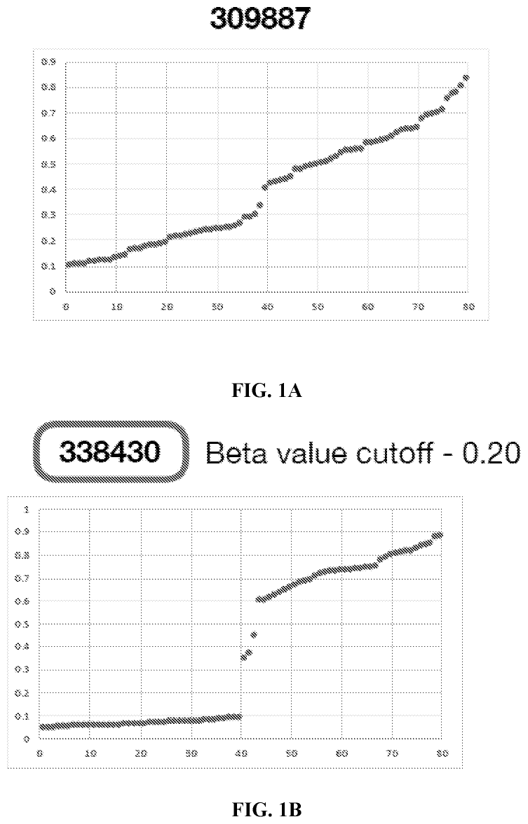

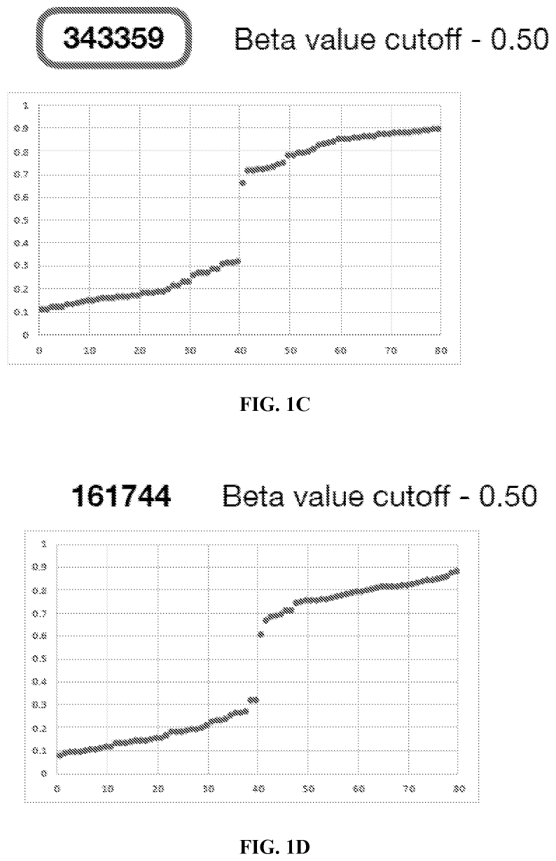

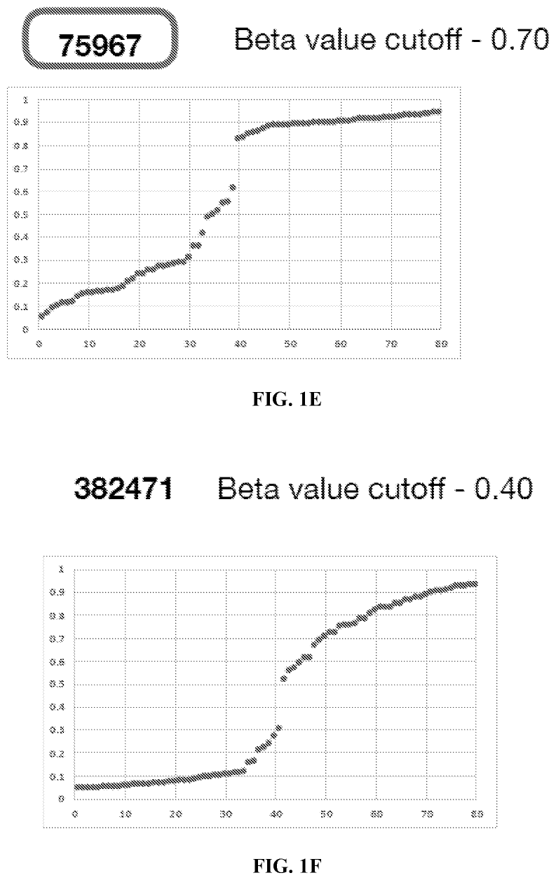

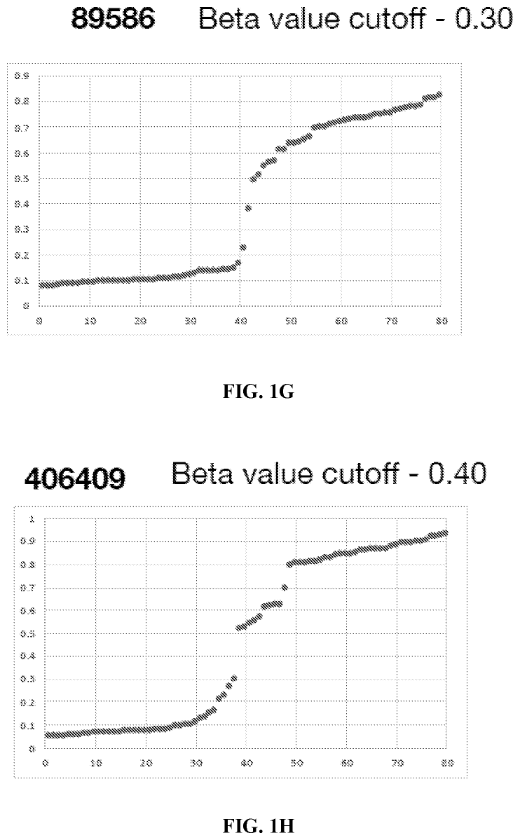

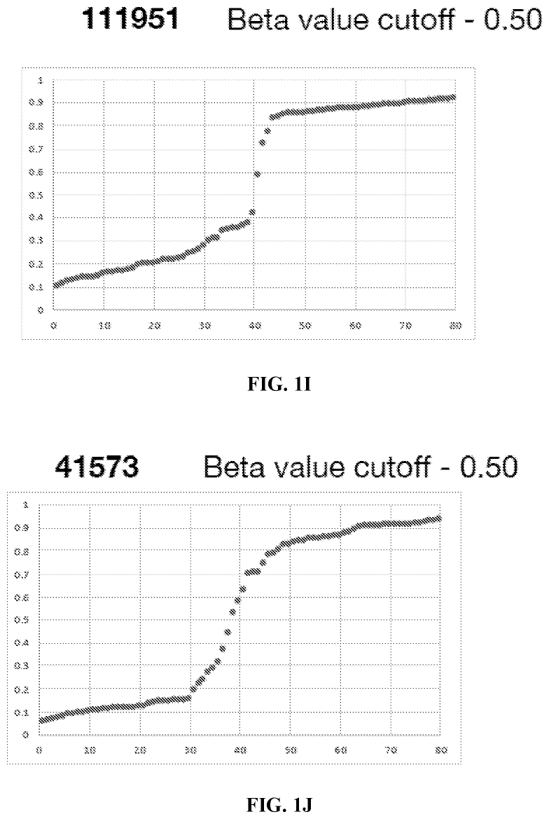

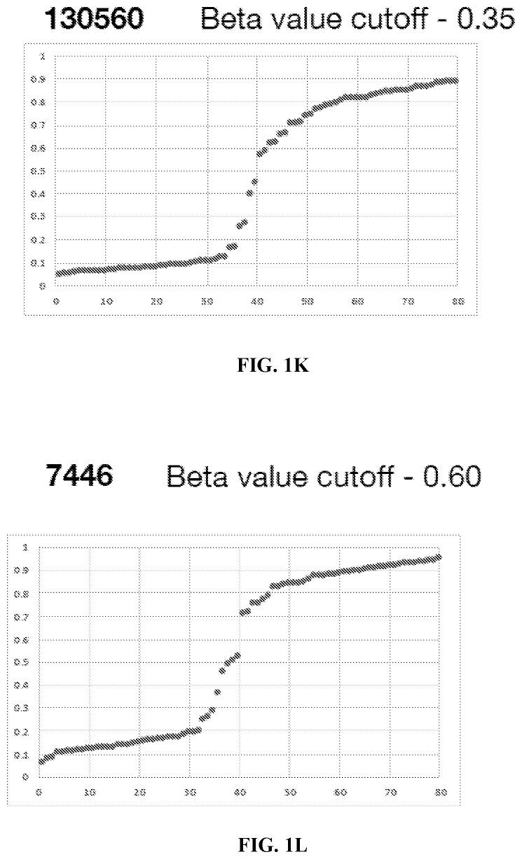

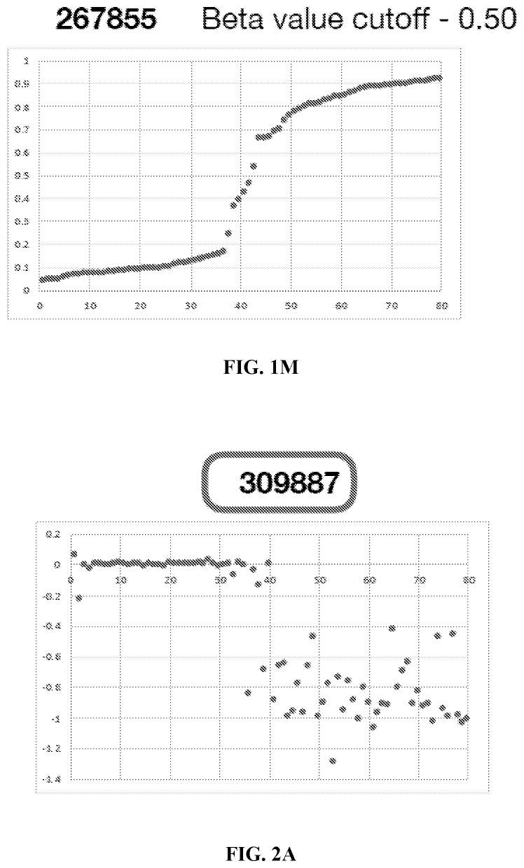

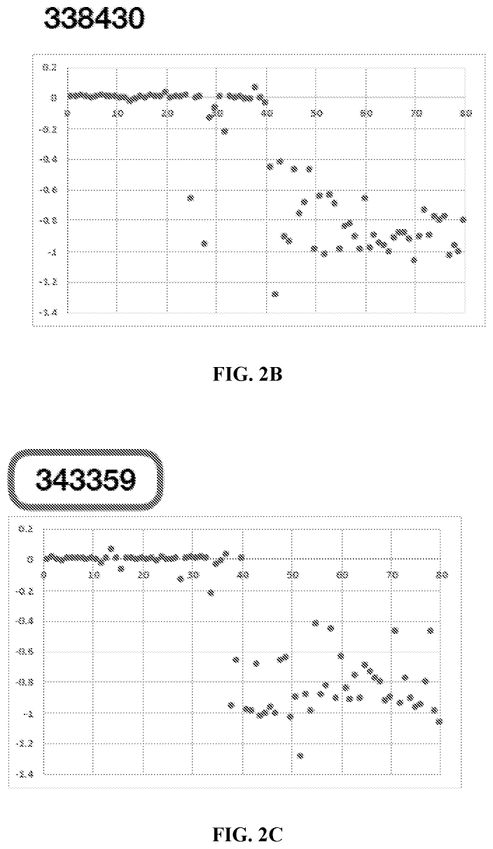

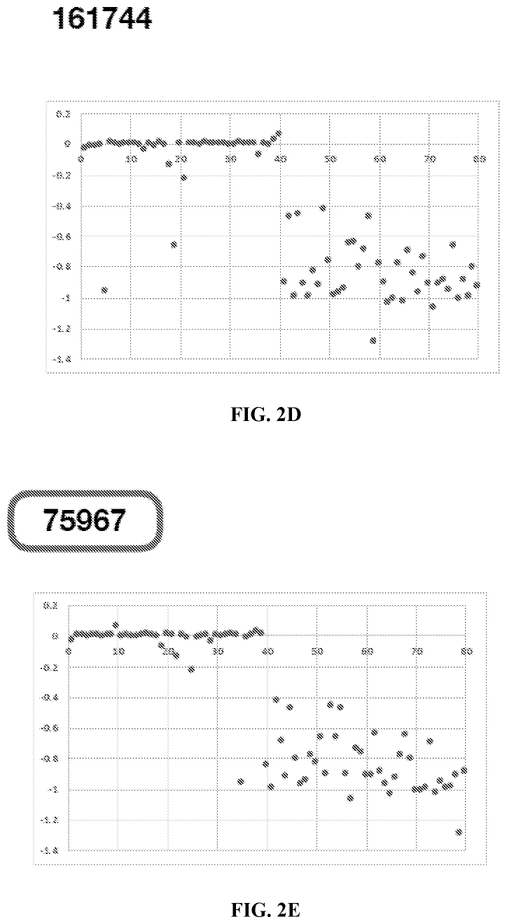

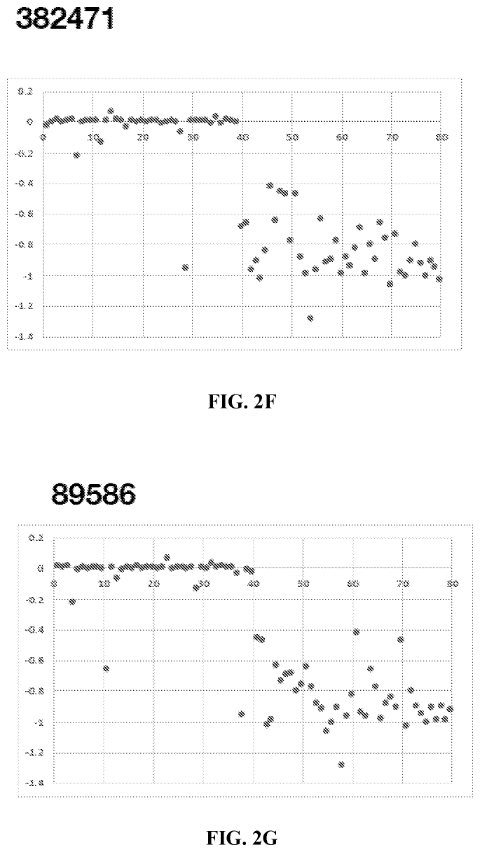

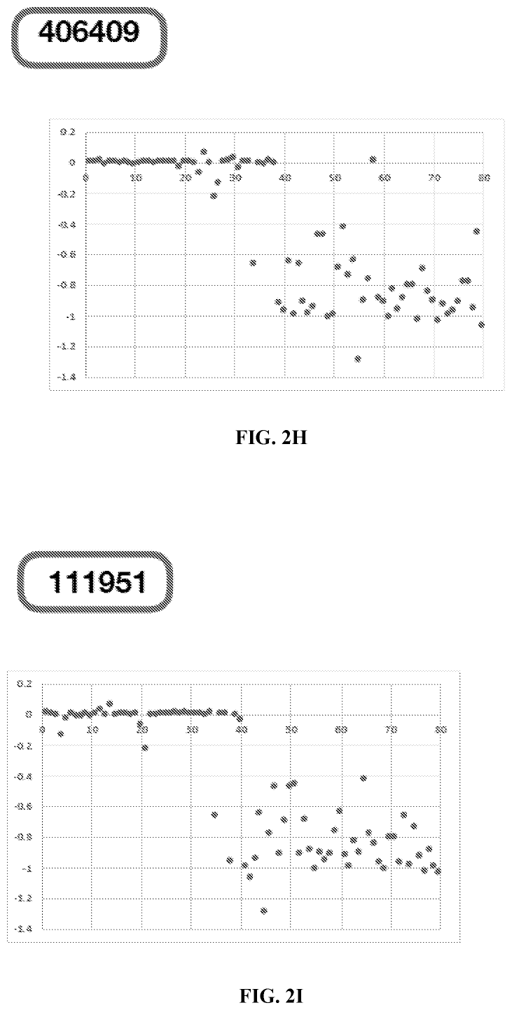

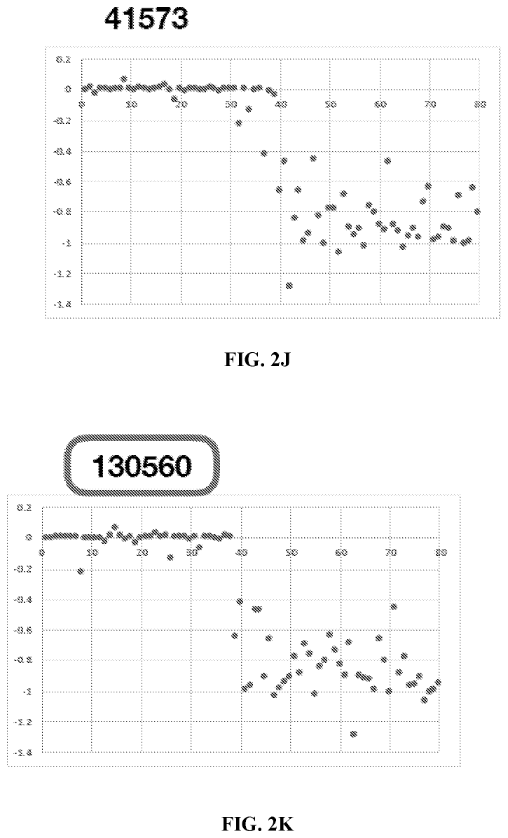

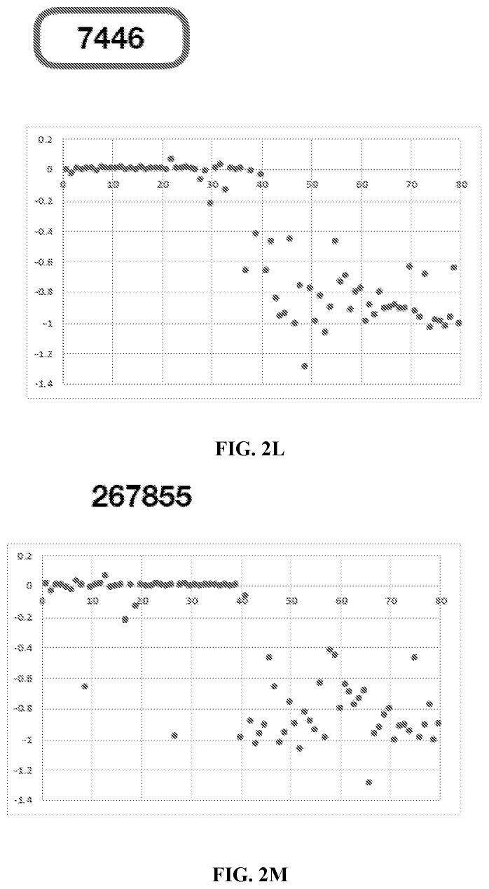

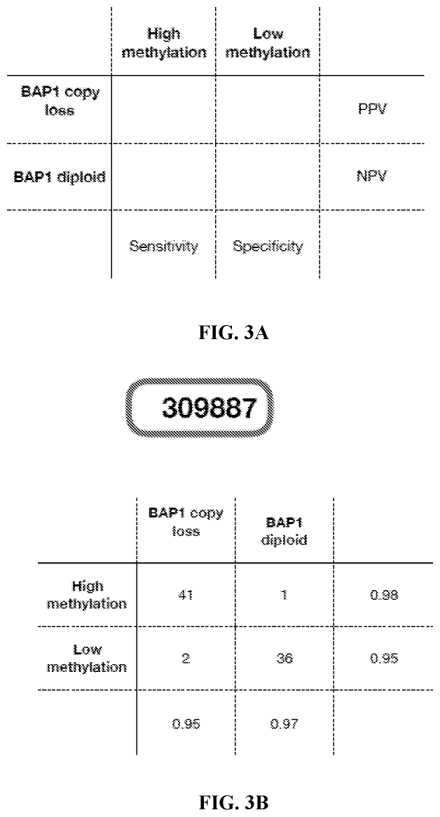

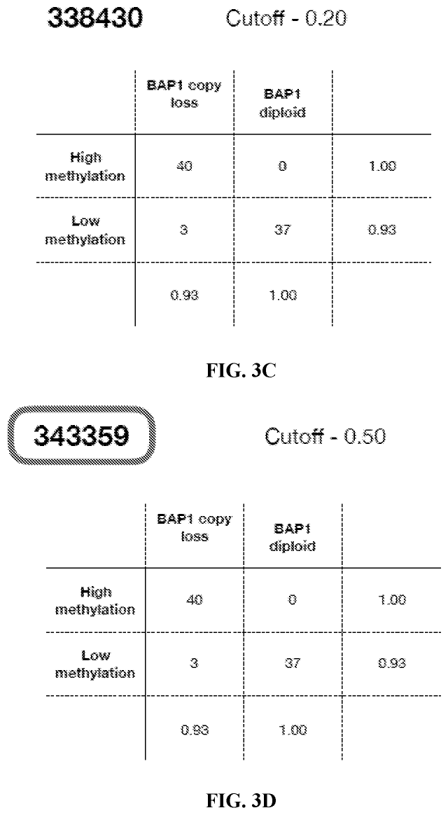

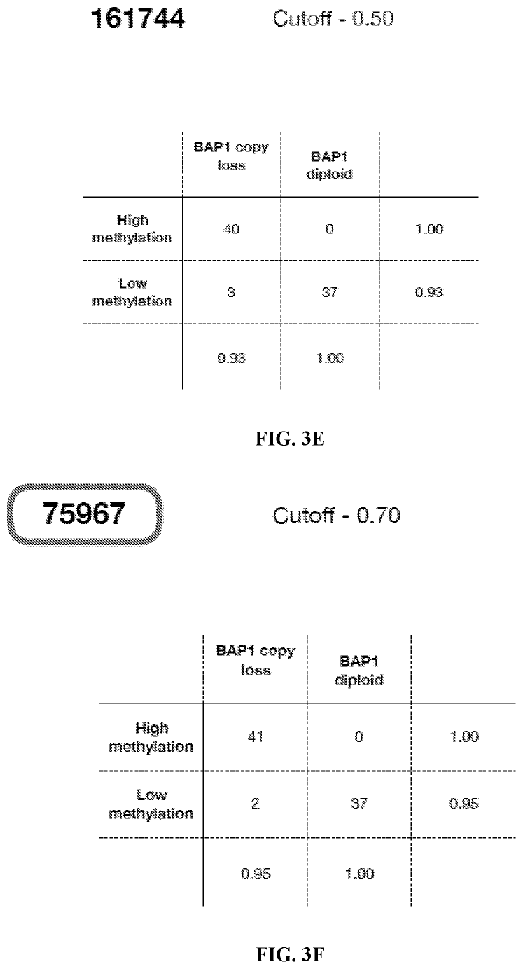

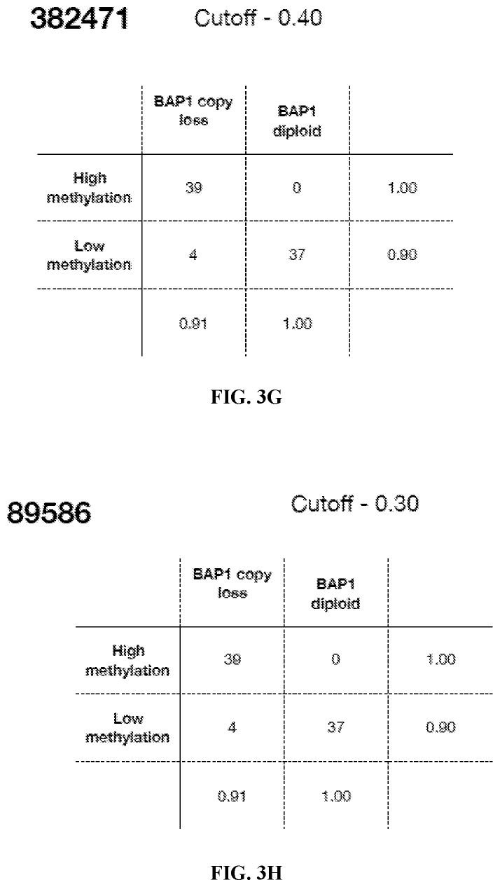

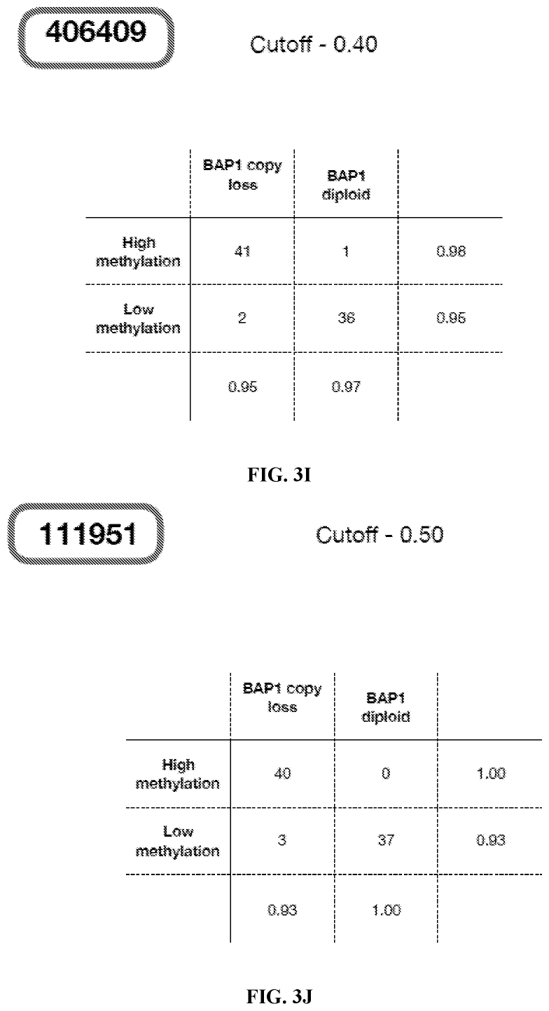

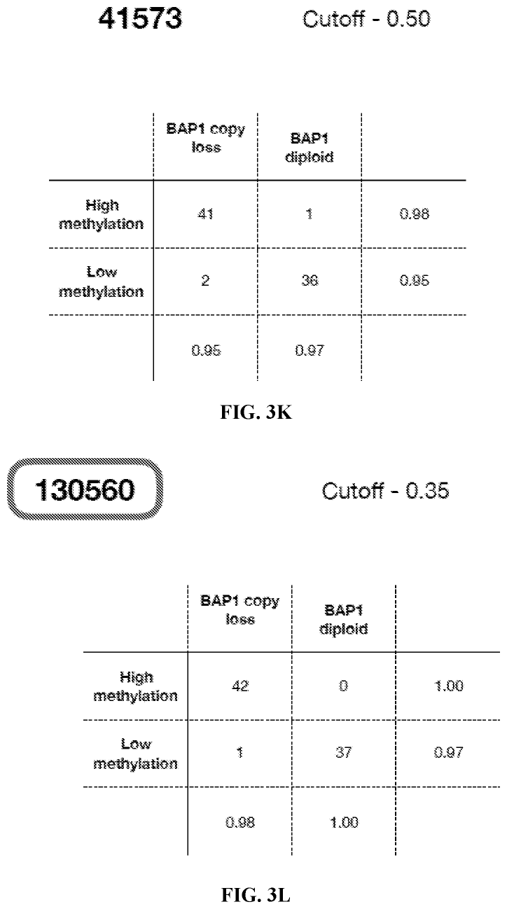

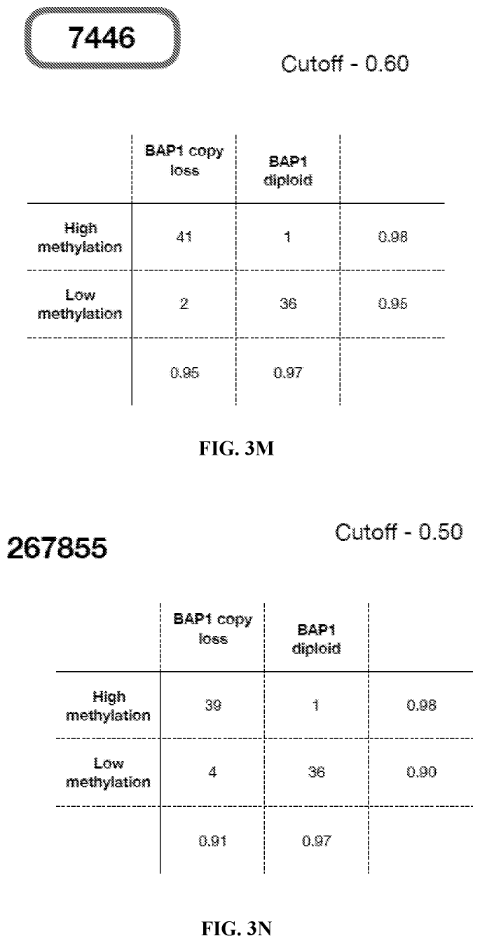

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2% and such ranges are included. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications may be referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this invention pertains.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a”, “an”, and “the” include plural references unless the context clearly dictates otherwise. For example, the term “a cell” includes a plurality of cells, including mixtures thereof.

As used herein, “and/or” refers to and encompasses any and all possible combinations of one or more of the associated listed items, as well as the lack of combinations when interpreted in the alternative (“or”).

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

In some embodiments, a subject has or is suspected of having a disease such cancer or neoplastic disorder, e.g., as UM. In a further embodiment, the subject is suspect of having a primary or metastatic uveal melanoma (UM) and/or is diagnosed with a primary or metastatic uveal melanoma. In a further embodiment, the subject is free of skin cutaneous melanoma (SKCM). In some embodiment, a subject is diagnosed with UM or is diagnosed as having a high probability of developing a UM. Such diagnostic methods are disclosed in, for example, Onken et al., Collaborative Ocular Oncology Group report number 1: prospective validation of a multi-gene prognostic assay in uveal melanoma. Ophthalmology 2012; 119: 1596-1603; and Field M G, Harbour J W. Recent developments in prognostic and predictive testing in uveal melanoma. Curr Opin Ophthalmol 2014; 25: 234-239.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at cancer.gov, last visited on May 1,2008. Typically, a patient is given a subsequent chemotherapy regimen because the patient did not show a positive clinical or sub-clinical response to the first line therapy or the first line therapy has stopped.

As used herein, a chemotherapy refers to a drug treatment that uses powerful chemicals to kill fast-growing cells in a subject. Non-limiting examples of a chemotherapy drugs include but are not limited to cyclophosphamide, methotrexate, 5-fluorouracil, doxorubicin, docetaxel, bleomycin, vinblastine, dacarbazine, mustine, vincristine, procarbazine, prednisolone, etoposide, cisplatin, capecitabine, methotrexate, vinorelbine, folinic acid, and oxaliplatin.

As used herein, a radiation therapy refers to a therapy using ionizing radiation (for example, an X-ray, a gamma ray, an electron beam, a proton beam, a neutron beam, or a heavy particle beam), generally as part of cancer treatment to control or kill malignant cells and normally delivered by a linear accelerator.

As used herein, the term “administer” and “administering” are used to mean introducing the therapeutic agent (e.g. polynucleotide, vector, cell, modified cell, population) into a subject. The therapeutic administration of this substance serves to attenuate any symptom, or prevent additional symptoms from arising. When administration is for the purposes of preventing or reducing the likelihood of developing an autoimmune disease or disorder, the substance is provided in advance of any visible or detectable symptom. Routes of administration include, but are not limited to, oral (such as a tablet, capsule or suspension), topical, transdermal, intranasal, vaginal, rectal, subcutaneous intravenous, intraarterial, intramuscular, intraosseous, intraperitoneal, intraocular, subconjunctival, sub-Tenon's, intravitreal, retrobulbar, intracameral, intratumoral, epidural and intrathecal.

The term “cell” as used herein may refer to either a prokaryotic or eukaryotic cell, optionally obtained from a subject or a commercially available source.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

An “effective amount” or “efficacious amount” refers to the amount of an agent or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

In one embodiment, the term “disease” or “disorder” as used herein refers to uveal melanoma (UM), a status of being diagnosed with UM, or a status of being suspect of having UM. In a further embodiment, UM includes (but is not limited to) choroidal melanoma, ciliary body melanoma, posterior uveal melanoma, or iris melanoma. Additionally or alternatively, UM may refer to a local UM, a metastatic UM (such as in liver, lung, and bones as well as subcutaneous metastasis), a non-metastatic UM, a primary UM, an advanced UM, an unresectable melanoma, or a recurrent UM. As used herein, an advanced UM refers to a UM which had progressed after receiving one or more of: the first line therapy, the second line therapy, or the third line therapy. In certain embodiments, the term “disease” or “disorder” as used herein refers to skin cutaneous melanoma (SCM), a status of being diagnosed with SCM, or a status of being suspect of having SCM.

As used herein, a “cancer” is a disease state characterized by the presence in a subject of cells demonstrating abnormal uncontrolled replication and may be used interchangeably with the term “tumor.” In certain embodiments, a cancer refers to a solid tumor and/or a non-solid cancer. Additionally or alternatively, a cancer may be a primary cancer or a metastasis.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound. In certain embodiments, the term “mRNA expression” refers to abundance of mRNA, i.e., mRNA amount or level.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, or a sample, separated from other DNAs or RNAs, or proteins or polypeptides, or cells or cellular organelles, or tissues or organs, or samples, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

As used herein, the term “risk” refers to the chance of a subject having or developing a disease (such as uveal melanoma) and/or a less favorable prognosis. In one embodiment, a less favorable prognosis comprises, or alternatively consists essentially of, or yet further consists of a decreased overall survival, and/or an increased probability of having a metastasis (for example in liver). Accordingly, a high-risk refers to more chance of having or developing a disease (such as a uveal melanoma) and/or a less favorable prognosis, while a low-risk refers to less chance of having or developing a disease (such as a uveal melanoma) and/or a less favorable prognosis (i.e., a more favorable prognosis).

As used herein, the term “a less favorable prognosis” is used interchangeably with poor prognosis, which comprises, or alternatively consists essentially of, or yet further consists of a decreased overall survival, and/or an increased probability of having a metastasis (for example in liver). Further, “more favorable prognosis” and “good prognosis” comprises, or alternatively consists essentially of, or yet further consists of an increased overall survival and/or a decreased probability of having a metastasis (such as in liver)

The term sets of (i) “high” “intermediate” and “low”; (ii) “more” “intermediate” and “less”; and/or (iii) “good” “intermediate” and “poor” refer to a relative differences in the recited matter in a descending order. In certain embodiments, these relative terms are used to identify a group and/or a property (such as methylation level, mRNA expression ratio, and/or risk) for other groups and/or properties (i.e., distinguishing groups or properties from each other) as defined herein.

An overall survival as used herein may refer to probability and/or duration of survival. Non-limiting examples of increased probability and/or duration of survival include increased overall survival (OS), increased progression free survival (PFS), increased disease free survival (DFS), increased time to tumor recurrence (TTR) and increased time to tumor progression (TTP). Non-limiting examples of decreased probability and/or duration of survival include decreased overall survival (OS), decreased progression free survival (PFS), decreased disease free survival (DFS), decreased time to tumor recurrence (TTR) and decreased time to tumor progression (TTP).

A “complete response” (CR) to a therapy defines patients with evaluable but non-measurable disease, whose tumor and all evidence of disease had disappeared.

A “partial response” (PR) to a therapy defines patients with anything less than complete response that were simply categorized as demonstrating partial response.

“Stable disease” (SD) indicates that the patient is stable.

“Progressive disease” (PD) indicates that the tumor has grown (i.e. become larger), spread (i.e. metastasized to another tissue or organ) or the overall cancer has gotten worse following treatment. For example, tumor growth of more than 20 percent since the start of treatment typically indicates progressive disease.

“Disease free survival” (DFS) indicates the length of time after treatment of a cancer or tumor during which a patient survives with no signs of the cancer or tumor.

“Non-response” (NR) to a therapy defines patients whose tumor or evidence of disease has remained constant or has progressed.

“Overall Survival” (OS) intends a prolongation in life expectancy as compared to naïve or untreated individuals or patients. In certain embodiments, an overall survival may refer to survival probability and/or duration.

“Progression free survival” (PFS) or “Time to Tumor Progression” (TTP) indicates the length of time during and after treatment that the cancer does not grow. Progression-free survival includes the amount of time patients have experienced a complete response or a partial response, as well as the amount of time patients have experienced stable disease.

“No Correlation” refers to a statistical analysis showing no relationship between the allelic variant of a polymorphic region or gene expression levels and clinical parameters.

“Tumor Recurrence” as used herein and as defined by the National Cancer Institute is cancer that has recurred (come back), usually after a period of time during which the cancer could not be detected. The cancer may come back to the same place as the original (primary) tumor or to another place in the body. It is also called recurrent cancer.

“Time to Tumor Recurrence” (TTR) is defined as the time from the date of diagnosis of the cancer to the date of first recurrence, death, or until last contact if the patient was free of any tumor recurrence at the time of last contact. If a patient had not recurred, then TTR was censored at the time of death or at the last follow-up.

“Relative Risk” (RR), in statistics and mathematical epidemiology, refers to the risk of an event (or of developing a disease) relative to exposure. Relative risk is a ratio of the probability of the event occurring in the exposed group versus a non-exposed group.

As used herein, the terms “stage I cancer,” “stage II cancer,” “stage III cancer,” and “stage IV” refer to the TNM staging classification for cancer. Stage I cancer typically identifies that the primary tumor is limited to the organ of origin. Stage II intends that the primary tumor has spread into surrounding tissue and lymph nodes immediately draining the area of the tumor. Stage III intends that the primary tumor is large, with fixation to deeper structures. Stage IV intends that the primary tumor is large, with fixation to deeper structures. See pages 20 and 21, CANCER BIOLOGY, 2^{nd} Ed., Oxford University Press (1987).

The term “blood” refers to blood which includes all components of blood circulating in a subject including, but not limited to, red blood cells, white blood cells, plasma, clotting factors, small proteins, platelets and/or cryoprecipitate. This is typically the type of blood which is donated when a human patent gives blood.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Cryoprotectants” are known in the art and include without limitation, e.g., sucrose, trehalose, and glycerol. A cryoprotectant exhibiting low toxicity in biological systems is generally used.

“Carriers” are known in the art and include inert (for example, a detectable agent or label) or active, such as an adjuvant, diluent, binder, stabilizer, buffers, salts, lipophilic solvents, preservative, adjuvant or the like and pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume.

Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, a biological sample, or a sample, is obtained from a subject. Exemplary samples include, but are not limited to, cell sample, tissue sample, tumor biopsy, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy, for example, from a melanoma or a UM. In certain embodiments, the sample is one or more of the following: a blood, a urine, an aqueous vitreous humor, a tumor biopsy, a liquid biopsy sample, an aqueous humor, a vitreous humor, a sample from primary or metastatic UM, a tumor tissue in freshly obtained specimens, a tumor tissue in paraffin embedded tissue, a frozen tumor tissue, a tumor tissue in fixative, cell free DAN and/or circulating tumor DNA in biological fluids.

As used herein, the term “mathematical equivalents” (i.e., “equivalents” for example, of a number, a value, or a numerical range) refers to various formats, methods, or means in describing the same properties (such as methylation level, mRNA expression ratio, and/or risk) as disclosed herein.

The term “BAP1” is used interchangeably with “BAP-1” and refers to BRCA1 Associated Protein 1 (which is also known as Ubiquitin carboxyl-terminal hydrolase BAP1) or a polynucleotide (such as a gene, a DNA, a mRNA or a hybrid thereof) encoding the same. The amino acid sequence, structure and other information relating to a Homo sapiens (human) BAP1 protein can be found at uniprot.org/uniprot/Q92560, which is incorporated herein in its entirety by reference. See UniProt Identifier Q92560-1 for an amino acid sequence of the BAP1 protein. Additionally, certain BAP-1 variants have been linked with a disease, such as E315A, G178V, A95D, C91W, F81V, S63C and I47F. The numbering of the amino acid as used herein is based on Q92560-1. Therefore, the BAP-1 protein variant(s), a polynucleotide encoding the variant and/or a gene in a subject encoding the variant are referred to herein as a BAP1 mutation. Accordingly, proteins, polynucleotides, and/or genes of BAP-1 which do not comprise or do not express such a variant, are referred to herein as a wildtype. In certain embodiments, a wildtype BAP-1 protein is a biological equivalent to the BAP-1 having the sequence identified as UniProt Q92560-1. The terms “equivalent” or “biological equivalent” are used interchangeably when referring to a particular molecule, biological, or cellular material and intend those having minimal homology while still maintaining desired structure or functionality. Non-limiting examples of equivalent polypeptides, include a polypeptide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide sequences, or a polypeptide which is encoded by a polynucleotide or its complement that hybridizes under conditions of high stringency to a polynucleotide encoding such polypeptide sequences. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity to the reference polynucleotide, e.g., the wild-type polynucleotide.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can be determined by incorporating them into clustalW (available at the web address: genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

As used herein, a methylation level can be quantified as a beta value, which is the average of methylated loci over non-methylated. i.e. a beta value of 0.1 means 10% of a specific genomic locus is methylated.

The term “CCNG2” is a polynucleotide (such as a gene, a DNA, an mRNA or any hybrid thereof) that encodes a Cyclin-G2 protein. The nucleotide sequence, amino acid sequence, structure and other information relating to CCNG2 and its Homo sapiens (human) protein can be found at uniprot.org/uniprot/Q16589 and www.genecards.org/cgi-bin/carddisp.pl?gene=CCNG2, each of which is incorporated herein in its entirety by reference.

The term “FKBP14” is a polynucleotide (such as a gene, a DNA, an mRNA or any hybrid thereof) that encodes a Peptidyl-prolyl cis-trans isomerase FKBP14 protein. The nucleotide sequence, amino acid sequence, structure and other information relating to FKBP14 and its Homo sapiens (human) FKBP14 protein can be found at uniprot.org/uniprot/Q9NWM8 and www.genecards.org/cgi-bin/carddisp.pl?gene=FKBP14, each of which is incorporated herein in its entirety by reference.

The term “JARID2” is a polynucleotide (such as a gene, a DNA, an mRNA or any hybrid thereof) that encodes Protein Jumonji. The nucleotide sequence, amino acid sequence, structure and other information relating to JARID2 and its Homo sapiens (human) protein can be found at uniprot.org/uniprot/Q92833 and www.genecards.org/cgi-bin/carddisp.pl?gene=JARID2, each of which is incorporated herein in its entirety by reference.

The term “TMEM173” which is known as “STING1” is a polynucleotide (such as a gene, a DNA, an mRNA or any hybrid thereof) that encodes Stimulator of interferon genes protein. The nucleotide sequence, amino acid sequence, structure and other information relating to TMEM173 and its Homo sapiens (human) protein can be found at uniprot.org/uniprot/Q86WV6 and www.genecards.org/cgi-bin/carddi sp.pl?gene=STING1, each of which is incorporated herein in its entirety by reference.

The term “PDE4B” is a polynucleotide (such as a gene, a DNA, an mRNA or any hybrid thereof) that encodes cAMP-specific 3′,5′-cyclic phosphodiesterase 4B. The nucleotide sequence, amino acid sequence, structure and other information relating to PDE4B and its Homo sapiens (human) protein can be found at uniprot.org/uniprot/Q07343 and www.genecards.org/cgi-bin/carddisp.pl?gene=PDE4B, each of which is incorporated herein in its entirety by reference.

The term “SH3D19” is a polynucleotide (such as a gene, a DNA, an mRNA or any hybrid thereof) that encodes SH3 domain-containing protein 19. The nucleotide sequence, amino acid sequence, structure and other information relating to SH3D19 and its Homo sapiens (human) protein can be found at uniprot.org/uniprot/Q5HYK7 and www.genecards.org/cgi-bin/carddisp.pl?gene=SH3D19, each of which is incorporated herein in its entirety by reference.

The term “SLC6A15” is a polynucleotide (such as a gene, a DNA, an mRNA or any hybrid thereof) that encodes Sodium-dependent neutral amino acid transporter B(0)AT2. The nucleotide sequence, amino acid sequence, structure and other information relating to SLC6A15 and its Homo sapiens (human) protein can be found at uniprot.org/uniprot/Q9H2J7 and www.genecards.org/cgi-bin/carddisp.pl?gene=SLC6A15, each of which is incorporated herein in its entirety by reference.

The term “LMCD1” is a polynucleotide (such as a gene, a DNA, an mRNA or any hybrid thereof) that encodes LIM and cysteine-rich domains protein 1. The nucleotide sequence, amino acid sequence, structure and other information relating to LMCD1 and its Homo sapiens (human) LMCD1 protein can be found at uniprot.org/uniprot/Q9NZU5. Information relating to the LMCD1 gene can be found at www.genecards.org/cgi-bin/carddisp.pl?gene=LMCD1 while the LMCD1-AS1 gene can be found at the www.genecards.org/cgi-bin/carddisp.pl?gene=LMCD1-AS1. Each of the webpage is incorporated herein in its entirety by reference.

The term “SENP6” is a polynucleotide (such as a gene, a DNA, an mRNA or any hybrid thereof) that encodes Sentrin-specific protease 6. The nucleotide sequence, amino acid sequence, structure and other information relating to SENP6 and its Homo sapiens (human) protein can be found at uniprot.org/uniprot/Q9GZR1 and www.genecards.org/cgi-bin/carddisp.pl?gene=SENP6, each of which is incorporated herein in its entirety by reference.

The term “NFIA” is a polynucleotide (such as a gene, a DNA, an mRNA or any hybrid thereof) that encodes Nuclear factor 1 A-type. The nucleotide sequence, amino acid sequence, structure and other information relating to NFIA and its Homo sapiens (human) protein can be found at uniprot.org/uniprot/Q12857 and www.genecards.org.chi-bin.carddisp.pl?gene=NFIA, each of which is incorporated herein in its entirety by reference.

The term “CTD-2207O23.12” refers to an uncharacterized gene located at Chromosome 19: 7,445,850-7,535,131 forward strand. See grch37.ensembl.org/Homo_sapiens/Gene/Summary?g=ENSG00000268861; r=19:7445850-7535131; t=ENST00000593531 for more information.

The term “ZNF358” is a polynucleotide (such as a gene, a DNA, an mRNA or any hybrid thereof) that encodes Zinc finger protein 358. The nucleotide sequence, amino acid sequence, structure and other information relating to ZNF358 and its Homo sapiens (human) protein can be found at uniprot.org/uniprot/Q9NW07 and www.genecards.org/cgi-bin/carddisp.pl?gene=ZNF358, each of which is incorporated herein in its entirety by reference.

The term “CCDC176”, which is also known as BBOF1, is a polynucleotide (such as a gene, a DNA, an mRNA or any hybrid thereof) that encodes Basal body-orientation factor 1. The nucleotide sequence, amino acid sequence, structure and other information relating to CCDC176 and its Homo sapiens (human) protein can be found at uniprot.org/uniprot/Q8NDO7 and www.genecards.org/cgi-bin/carddisp.pl?gene=BBOF1, each of which is incorporated herein in its entirety by reference.

The term “ENTPD5” is a polynucleotide (such as a gene, a DNA, an mRNA or any hybrid thereof) that encodes Ectonucleoside triphosphate diphosphohydrolase 5. The nucleotide sequence, amino acid sequence, structure and other information relating to ENTPD5 and its Homo sapiens (human) protein can be found at uniprot.org/uniprot/075356 and www.genecards.org/cgi-bin/carddisp.pl?gene=ENTPD5, each which is incorporated herein in its entirety by reference.

The term “DTWD1” is a polynucleotide (such as a gene, a DNA, an mRNA or any hybrid thereof) that encodes DTW domain-containing protein 1. The nucleotide sequence, amino acid sequence, structure and other information relating to DTWD1 and its Homo sapiens (human) protein can be found at uniprot.org/uniprot/Q8N5C7 and www.genecards.org/cgi-bin/carddisp.pl?gene=DTWD1, each of which is incorporated herein in its entirety by reference.

The term “FAM227B” is a polynucleotide (such as a gene, a DNA, an mRNA or any hybrid thereof) that encodes Family With Sequence Similarity 227 Member B. The nucleotide sequence, amino acid sequence, structure and other information relating to FAM227B and its Homo sapiens (human) protein can be found at uniprot.org/uniprot/Q96M60 and www.genecards.org/cgi-bin/carddisp.pl?gene=FAM227B, each of which is incorporated herein in its entirety by reference.

The term “SESN1” is a polynucleotide (such as a gene, a DNA, an mRNA or any hybrid thereof) that encodes Sestrin-1. The nucleotide sequence, amino acid sequence, structure and other information relating to SESN1 and its Homo sapiens (human) protein can be found at uniprot.org/uniprot/Q9Y6P5 and www.genecards.org/cgi-bin/carddisp.pl?gene=SESN1, each of which is incorporated herein in its entirety by reference.

The term “ENPP2” is a polynucleotide (such as a gene, a DNA, an mRNA or any hybrid thereof) that encodes Ectonucleotide pyrophosphatase/phosphodiesterase family member 2. The nucleotide sequence, amino acid sequence, structure and other information relating to ENPP2 and its Homo sapiens (human) protein can be found at uniprot.org/uniprot/and www.genecards.org/cgi-bin/carddisp.pl?gene=ENPP2, each of which is incorporated herein in its entirety by reference.

As used herein chromosome 3 monosomy refers to an about 100%, or at least about 90%, or at least about 80%, or at least about 70%, or at least about 60%, or at least about 50%, or at least about 40%, or at least about 30%, or at least about 20%, or at least about 10%, or at least about 5% loss of heterozygosity in chromosome 3, which has been linked with less favorable prognosis of a subject having uveal melanoma. A less than 100% loss of heterozygosity is referred to herein as a partial monosomy, while an about 0%, or less than about 1%, or less than about 2%, or less than about 3%, or less than about 4%, or less than about 5%, or less than about 10%, or less than about 20% loss of heterozygosity is referred to herein as a disomy.

As used herein, the term “heterozygosity” refers to the condition of having two different alleles at a locus.

### Modes of Carrying Out the Disclosure

Uveal melanoma is an intraocular malignancy that arises from melanocytes of the choroid, ciliary body, and iris of the eye. Yang, J. et al. (2018) Ther Adv Med Oncol., 10. In clinical practice prognostication is important to inform patients about their prognosis (chance of developing metastasis and overall survival), guiding metastatic surveillance, guiding treatment and enrollment into clinical trials.

Applicants have found that a high methylation level on the gene BAP1 at a single locus (CGI coordinates, CGI:chr3:52409661-52410118) can predict patient survival and can be used to estimate prognostic classes. BRCA1-associated protein-1 (BAP1) gene, located at 3p21, encodes a deubiquitinating enzyme involved in the removal of ubiquitin from proteins. Alteration on BAP1 mRNA expression or mutations on the BAP1 gene have been associated with UM metastasis since it naturally shows tumor suppressor activity.

On another note, Applicants have also found that an assay that evaluates the relative expression of two genes from bulk sequencing and identified a subpopulation of cells that accounts for poor survival outcomes by looking at the ratio of mRNA expression of two genes JARID2 and TMEM173.

Applicants analyzed the methylation signature obtained from 80 uveal specimens. These underwent whole exome sequencing, RNA sequencing and have associated clinical findings including survival. This allowed Applicants to isolate a set of genomic loci where methylation is highly associated with poor survival and can reflect BAP1 and chromosome 3 copy number status and BAP1 mutations. Each of these loci can independently predict these outcomes at a high confidence. The decision to pick more than one locus is to increase the test sensitivity when applied to liquid biopsies.

Applicant's research has identified significant intratumor heterogeneity whereby each tumor contained a heterogeneous admixture of cells that resembled both phenotypes (high risk and low risk) to varying degrees. Further, Applicant identified high-risk UM with good, intermediate and poor prognosis; identified tumors with partial or full monosomy 3, as well as disomy 3, identified tumors with pathogenic mutations in BAP1, as well as those with wild type BAP1 gene, identified the preponderance of tumor cells with high-risk features in UM, predicted overall survival of patients with UM (primary and/or metastatic), and predicted the chance of metastasis, usually to the liver, lungs and/or distant organs. These markers comprises one or more of the following:
(1) high relative DNA methylation of the following genes especially at the loci identified in Table 7: (a) BAP1, and (b) In addition to LMCD1 LMCD1-AS1, SENP6, CCNG2, NFIA, CTD-2207O23.12/ZNF358, CTD-2207O23.12/ZNF358, CCDC176/ENTPD5, DTWD1/FAM227B, PDE4B, SESN1 and ENPP2;
(2) mRNA expression of JARID2;
(3) mRNA expression of TMEM173 (STING); or
(4) Ratio of expression of JARID2 relative to TMEM173 (STING) or TMEM173 (STING) relative to JARID2.

### Methods of Treatment

This disclosure provides methods for treating uveal melanoma in a subject in need thereof, the subject having been selected for the treatment by a method comprising, or alternatively consisting essentially of, or yet further consisting of detection of high methylation levels at one or more of: BAP-1, CCNG2 and/or FKBP14 and/or measuring the mRNA expression of JARID2 and TMEM173 and determining ratio of mRNA expression JARID2 to TMEM173 in a sample isolated from the subject, the method comprising, or alternatively consisting essentially of, or yet further consisting of administering an effective amount of one or more of: an anti-cancer drug, immunotherapy, chemotherapy, and/or radiation therapy, thereby treating the subject. In one aspect, the method comprising, or alternatively consisting essentially of, or yet further consisting of detection further comprises, or alternatively consists essentially of, or yet further consists of detecting high methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7. In another aspect, the method of detection comprises, or alternatively consists essentially of, or yet further consists of detecting high methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or measuring the mRNA expression of JARID2 and TMEM173 and determining ratio of mRNA expression JARID2 to TMEM173 in a sample isolated from the subject.

In a further aspect, the subject is selected when the ratio of mRNA expression JARID2 to TMEM173 is less than about 1. In a particular aspect, the subject is selected when the ratio of mRNA expression JARID2 to TMEM173 is less than about 1, or alternatively less than about 0.95, or alternatively less than about 0.90, or alternatively less than about 0.85, or alternatively less than about 0.80, or alternatively less than about 0.75, or alternatively less than about 0.70, or alternatively less than about 0.65, or alternatively less than about 0.60, or alternatively less than about 0.55, or alternatively less than about 0.50, or alternatively less than about 0.45, or alternatively less than about 0.40, or alternatively less than about 0.35, or alternatively less than about 0.30, or alternatively less than about 0.25, or alternatively less than about 0.20, or alternatively less than about 0.15, or alternatively less than about 0.10, or alternatively less than about 0.05, or alternatively less than about 0.04, or alternatively less than about 0.03, or alternatively less than about 0.02, or alternatively less than about 0.01.

Provided are methods for treating a high-risk uveal melanoma in a subject comprising, or alternatively consisting essentially of, or yet further consisting of administering an effective amount of one or more of: an anti-cancer drug, immunotherapy, chemotherapy, and/or radiation therapy selective for the uveal melanoma (for example, the high-risk uveal melanoma), thereby treating the subject. In one embodiment, a sample isolated from the subject has one or more of the following: a high methylation levels at one or more of: BAP-1, CCNG2 and/or FKBP14; a low mRNA expression ratio of JARID2 to TMEM173; a high methylation level at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2; and/or a high methylation level at one or more of the methylation loci identified in Table 2 and/or 7.

In another embodiment, the method comprises, or alternatively consists essentially of, or yet further consists of detecting high methylation levels at one or more of: BAP-1, CCNG2, FKBP14, PDE4B, SH3D19, SLC6A15 LMCD1, LMCD1-AS1, SENP6, NFIA, CTD-2207023.12, ZNF358, CCDC176, ENTPD5, DTWD1, FAM227B, SESN1, ENPP2 and/or methylation loci identified in Table 2 and/or 7, and/or determining a low ratio of mRNA expression JARID2 to TMEM173 in a sample isolated from the subject.

In a further embodiment, the method (for example, the detection step of the method) comprises, or alternatively consists essentially of, or yet further consists of measuring the mRNA expression of JARID2 and TMEM173 and determining a low ratio of mRNA expression JARID2 to TMEM173 in a sample isolated from the subject.

Also provided is a method for treating a low-risk uveal melanoma in a subject comprising, or alternatively consisting essentially of, or yet further consisting of administering an effective amount of one or more of: an anti-cancer drug, immunotherapy, chemotherapy, and/or radiation therapy selective for the uveal melanoma (for example, the low-risk uveal melanoma), thereby treating the subject. In one embodiment, a sample isolated from the subject has one or more of the following: a low methylation levels at one or more of: BAP-1, CCNG2 and/or FKBP14; a high mRNA expression ratio of JARID2 to TMEM173; a low methylation level at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2; and/or a low methylation level at one or more of the methylation loci identified in Table 2 and/or 7.

In a further embodiment, the method (for example, the detection step of the method) comprises, or alternatively consists essentially of, or yet further consists of measuring the mRNA expression of JARID2 and TMEM173 and determining a high ratio of mRNA expression JARID2 to TMEM173 in a sample isolated from the subject.

Further provided is a method for treating an intermediate-risk uveal melanoma in a subject comprising, or alternatively consisting essentially of, or yet further consisting of administering an effective amount of one or more of: an anti-cancer drug, immunotherapy, chemotherapy, and/or radiation therapy selective for the uveal melanoma (for example, the intermediate-risk uveal melanoma), thereby treating the subject. In one embodiment, a sample isolated from the subject has one or more of the following: an intermediate methylation levels at one or more of: BAP-1, CCNG2 and/or FKBP14; an intermediate mRNA expression ratio of JARID2 to TMEM73; an intermediate methylation level at one or more of: PDE4B, SH3D19, SLC6A15 LMCD1, LMCD1-AS1, SENP6, NFIA, CTD-2207023.12, ZNF358, CCDC176, ENTPD5, DTWD1, FAM227B, SESN1, ENPP2; and/or an intermediate methylation level at one or more of the methylation loci identified in Table 2 and/or 7.

In a further embodiment, the method (for example, the detection step of the method) comprises, or alternatively consists essentially of, or yet further consists of measuring the mRNA expression of JARID2 and TMEM73 and determining an intermediate ratio of mRNA expression JARID2 to TMEM173 in a sample isolated from the subject. As used herein, “treating” uveal melanoma in a subject in need thereof refers to (1) preventing the symptoms of uveal melanoma or uveal melanoma from occurring in a subject that is predisposed or does not yet display symptoms of the disease; (2) inhibiting the disease or arresting its development; or (3) ameliorating or causing regression of uveal melanoma or the symptoms of uveal melanoma. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition caused by uveal melanoma (including uveal melanoma itself), stabilized (i.e., not worsening) state of a condition caused by uveal melanoma (including uveal melanoma itself), delay or slowing of condition caused by uveal melanoma (including uveal melanoma itself), progression, amelioration or palliation of condition caused by uveal melanoma (including uveal melanoma itself), states and remission (whether partial or total), whether detectable or undetectable. When the disease is uveal melanoma, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

In one embodiment, the treatment is administering an anti-cancer drug, immunotherapy, chemotherapy, radiation therapy, and any combination thereof to a subject. In one embodiment, and anti-cancer drug and/or an immunotherapy drug (which is an anti-cancer drug via acting on the immune system to treat a cancer such as antibody therapy and immune checkpoint inhibitors) is used, including but not limited to, IMCgp100 (Tebentafusp™), Nivolumab, ipilimumab, dabrafenib, trametinib, a PD-1 checkpoint inhibitor, an anti-PD-1 agent (such as an antibody or an equivalent thereof), an anti-PD-L1 agent (such as an antibody or an equivalent thereof), an immune checkpoint inhibitor, a BRAF/MEK-targeted therapy, an FAK (Focal adhesion kinase) inhibitor, a MEK (Mitogen-activated protein kinase kinase) inhibitor, and/or any combination thereof. See, for example, Kaštelan S et al., Curr Med Chem. 2020; 27(8):1350-1366 and Journal of the National Comprehensive Cancer Network J Natl Compr Canc Netw 16, 5S; 10.6004/jnccn.2018.0042. In certain embodiments, one or more of the treatments is suitable for treating a subject as described herein. In one embodiment, one or more of the treatments suitable for treating one or more of the following: a high-risk uveal melanoma, an intermediate-risk uveal melanoma, a low-risk uveal melanoma, a subject having a less favorable prognosis, a subject having an intermediate prognosis, or a subject having a more favorable prognosis.

In one embodiment, the BAP-1 methylation levels in the methods described herein are detected at the methylation loci identified in Tables 2, 3, 4 and/or 5. In one particular embodiment, BAP-1 methylation levels in the methods described herein are detected at the methylation locus CGI:chr3:52409661-52410118.

### Ratio of mRNA Expression JARID2 to TMEM173

In certain embodiments of these treatment methods as well as other methods and/or embodiments in this disclosure, mRNA expression levels of JARID2 to TMEM173 of samples are measured, determined, and then grouped into two groups based on the ratios of mRNA expression JARID2 to TMEM173, i.e., high ratio of mRNA expression JARID2 to TMEM173 group and low ratio of mRNA expression JARID2 to TMEM173 group. All samples in the high ratio (of JARID2 mRNA to TMEM173 mRNA) group comprises a ratio of mRNA expression JARID2 to TMEM173 higher than those in the low ratio group.

In certain embodiments of these treatment methods as well as other methods and/or embodiments in this disclosure, the low ratio of mRNA expression JARID2 to TMEM173 is less than about 100, or alternatively less than about 90, or alternatively less than about 80, or alternatively less than about 70, or alternatively less than about 60, or alternatively less than about 50, or alternatively less than about 40, or alternatively less than about 30, or alternatively less than about 25, or alternatively less than about 20, or alternatively less than about 15, or alternatively less than about 10, or alternatively less than about 9, or alternatively less than about 8, or alternatively less than about 7, or alternatively less than about 6, or alternatively less than about 5, or alternatively less than about 4, or alternatively less than about 3, or alternatively less than about 2.5, or alternatively less than about 2, or alternatively less than about 1.5, or alternatively less than about 1, or alternatively less than about 0.95, or alternatively less than about 0.90, or alternatively less than about 0.85, or alternatively less than about 0.80, or alternatively less than about 0.75, or alternatively less than about 0.70, or alternatively less than about 0.65, or alternatively less than about 0.60, or alternatively less than about 0.55, or alternatively less than about 0.50, or alternatively less than about 0.45, or alternatively less than about 0.40, or alternatively less than about 0.35, or alternatively less than about 0.30, or alternatively less than about 0.25, or alternatively less than about 0.20, or alternatively less than about 0.15, or alternatively less than about 0.10, or alternatively less than about 0.05, or alternatively less than about 0.04, or alternatively less than about 0.03, or alternatively less than about 0.02, or alternatively less than about 0.01. In a further embodiment, a high ratio of mRNA expression JARID2 to TMEM173 is a ratio other than any one of the above-identified low ratio. In one embodiment, a high ratio of mRNA expression JARID2 to TMEM173 is about 1 or greater than about 1 while a low ratio of mRNA expression JARID2 to TMEM173 is less than about 1.

In a further embodiment, the high and low ratio groups are divided by one ratio that divides a ratio distribution of a control population ordered by the ratio values into two groups, each containing a half of the population. In one embodiment, the control population comprises or alternatively consists essentially of, or yet further consists of samples isolated from a subject diagnosed with or suspect of having a disease (such as a uveal melanoma). In another embodiment, the control population comprises or alternatively consists essentially of, or yet further consists of samples isolated from a subject free of a disease (such as a uveal melanoma). In yet another embodiment, the control population comprises or alternatively consists essentially of, or yet further consists of samples isolated from a subject who may or may not have a disease (such as a uveal melanoma). In a particular embodiment, the control population comprises or alternatively consists essentially of, or yet further consists of the samples used in the Examples. In a further embodiment, analysis on the samples used in the Examples provide two groups divided by a median value of a control population.

In certain embodiments of these treatment methods as well as other methods and/or embodiments in this disclosure, mRNA expression levels of JARID2 to TMEM173 of samples are measured, determined, and then grouped into three groups based on the ratios of mRNA expression JARID2 to TMEM173, i.e., high ratio of mRNA expression JARID2 to TMEM173 group (referred to herein as the high ratio group), intermediate ratio of mRNA expression JARID2 to TMEM173 (referred to herein as the intermediate ratio group), and low ratio of mRNA expression JARID2 to TMEM173 (referred to herein as the low ratio group). It would be understood by one of skill in the art that the samples in the high ratio group comprises a ratio of mRNA expression JARID2 to TMEM173 higher than those in the intermediate and/or low ratio group, and the samples in the intermediate ratio group comprises a ratio of mRNA expression JARID2 to TMEM173 higher than those in the low ratio group.

In a further embodiment, the high, intermediate and low ratio groups are divided by two ratios that divide a ratio distribution of a control population ordered by the ratio values into three parts, each containing a third of the population. Such two ratios and/or three divided groups are referred to herein as a tertile (or tertile points and tertile groups, respectively). In one embodiment, the control population comprises or alternatively consists essentially of, or yet further consists of samples isolated from a subject diagnosed with or suspect of having a disease (such as a uveal melanoma). In another embodiment, the control population comprises or alternatively consists essentially of, or yet further consists of samples isolated from a subject free of a disease (such as a uveal melanoma). In yet another embodiment, the control population comprises or alternatively consists essentially of, or yet further consists of samples isolated from a subject who may or may not have a disease (such as a uveal melanoma). In a particular embodiment, the control population comprises or alternatively consists essentially of, or yet further consists of the samples used in the Examples.

In yet a further embodiment, four groups (for example, high, intermediate high, intermediate low, and low) are divided by three ratios based on the ratio values, each group containing a fourth of the population. In one embodiment, analysis on the samples used in the Examples provide four groups as JARID2/STING mRNA ratio greater than about 2 (high), about 1 to about 2 (intermediate high), about 0.3 to about 1 (intermediate low) and less than 0.3 (low). Accordingly, in certain embodiments of the treatment methods as well as other methods and/or embodiments in this disclosure, the term “intermediate” may be further specified as “intermediate high” and/or “intermediate low”.

In certain embodiments of these treatment methods as well as other methods and/or embodiments in this disclosure, a low ratio of mRNA expression JARID2 to TMEM173 is less than about 1, less than about 2 or less than about 0.3. A high ratio of mRNA expression JARID2 to TMEM173 is (a) about 1 and/or greater than about 1, (b) about 2 and/or greater than about 2, or (c) about 0.3 and/or less than about 0.3. In one embodiment, an intermediate ratio of mRNA expression JARID2 to TMEM173 is from about 0.3 to about 1, from about 0.3 to about 2, or from about 1 to about 2. In an alternative embodiment, a high ratio further comprises, or alternatively consists essentially of, or yet further consists of from about 0.3 to about 1, from about 0.3 to about 2, or from about 1 to about 2.

Additionally, one of skill in the art would have no difficulty in converting a ratio or a ratio range of mRNA expression JARID2 to TMEM173 to a corresponding ratio or ratio range of mRNA expression TMEM173 to JARID2, a corresponding percentage or a percentage range of TMEM173 mRNA over the total mRNAs of TMEM173 and JARID2, a corresponding percentage or a percentage range of JARID2 mRNA over the total mRNAs of TMEM173 and JARID2, or a mathematical equivalent thereof. Each of such corresponding ratios, ratio ranges, percentages or percentage ranges is also included as embodiments of the methods, compositions and kits in the disclosure.

### Methylation Levels

In certain embodiments of these treatment methods as well as other methods and/or embodiments in this disclosure, methylations are detected and then grouped into two groups based on the detected levels, i.e., high methylation level group and low methylation level group. All samples in the high methylation level group comprises a methylation level higher than those in the low high methylation level group.

Methods of determining DNA methylation are well-known in the art. Non-limiting examples of methods employed to determine DNA methylation can be found in Kurdyukov, S., & Bullock, M. (2016) Biology, 5(1), 3, which include HPLC-UV (high performance liquid chromatography-ultraviolet), LC-MS/MS (Liquid chromatography coupled with tandem mass spectrometry), ELISA-Based methods (enzyme-linked immunosorbent assay based methods), LINE-1 (long interspersed nuclear elements-1) and Pyrosequencing, AFLP (PCR-based amplification fragment length polymorphism) and RFLP (PCR-based restriction fragment length polymorphism), LUMA (luminometric methylation assay), Bisulfite Sequencing, Array or Bead Hybridization, Methyl-Sensitive Cut Counting: Endonuclease Digestion Followed by Sequencing, Bead Array, PCR and Sequencing, Pyrosequencing, Methylation-Specific PCR, PCR with High Resolution Melting and/or COLD-PCR for the Detection of Unmethylated Islands.

As used herein, a methylation level may be detected and/or presented as a beta value (β), which is a ratio of methylated alleles over the total of methylated and unmethylated alleles. Beta values are between 0 and 1 with 0 being unmethylated and 1 fully methylated.

Additionally, one of skill in the art would have no difficulty in quantifying a methylation level, presenting it using a parameter other than beta value as used herein (such as an M-value which is calculated as the log₂ ratio of the intensities of methylated probe versus unmethylated probe), and corresponding a methylation level and/or a methylation level range presented by such parameter to that presented using beta values. See, for example, Du, P., Zhang, X., Huang, C. et al. Comparison of Beta-value and M-value methods for quantifying methylation levels by microarray analysis. BMC Bioinformatics 11, 587 (2010). doi.org/10.1186/1471-2105-11-587. Any methylation levels or ranges corresponding to those recited herein using beta values are also included in this disclosure in any of the methods, embodiments, or kits as described herein.

In certain embodiments of these treatment methods as well as other methods and/or embodiments in this disclosure, the low methylation level is detected as a beta value or beta value range of less than about 1, or alternatively less than about 0.95, or alternatively less than about 0.90, or alternatively less than about 0.85, or alternatively less than about 0.80, or alternatively less than about 0.75, or alternatively less than about 0.70, or alternatively less than about 0.65, or alternatively less than about 0.60, or alternatively less than about 0.55, or alternatively less than about 0.50, or alternatively less than about 0.45, or alternatively less than about 0.40, or alternatively less than about 0.35, or alternatively less than about 0.30, or alternatively less than about 0.25, or alternatively less than about 0.20, or alternatively less than about 0.15, or alternatively less than about 0.10, or alternatively less than about 0.05, or alternatively less than about 0.04, or alternatively less than about 0.03, or alternatively less than about 0.02, or alternatively less than about 0.01. In a further embodiment, a high methylation level is a level other than any one of the above-identified low levels, with the proviso that a methylation level is detected with a beta value from 0 to 1. In one embodiment, a high methylation level comprises a beta value of about 0.3 or greater while a low methylation level comprises a beta value of less than about 0.3.

In certain embodiments of these treatment methods as well as other methods and/or embodiments in this disclosure, methylations are detected and then grouped into three groups based detected levels, i.e., high methylation level group, intermediate methylation level group and low methylation level group. All samples in the high methylation level group comprises a methylation level higher than those in the intermediate and/or low methylation level group, while all samples in the intermediate methylation level group comprises a methylation level higher than those in the low high methylation level group. In one embodiment, the high methylation level group having a methylation level detected with a beta value from about 0.75 to about 1, the intermediate methylation level group having a methylation level detected with a beta value from about 0.25 to lower than about 0.75, while the low methylation level group having a methylation level detected with a beta value from about 0 to lower than about 0.25.

In a further embodiment, the high, intermediate and low methylation level groups are divided by two ratios that divide an ratio distribution of a control population ordered by the detected levels into three parts, each containing a third of the population. Such two ratios and/or three divided groups are referred to herein as a tertile (or tertile points and tertile groups, respectively). In one embodiment, the control population comprises or alternatively consists essentially of, or yet further consists of samples isolated from a subject diagnosed with or suspect of having a disease (such as a uveal melanoma). In another embodiment, the control population comprises or alternatively consists essentially of, or yet further consists of samples isolated from a subject free of a disease (such as a uveal melanoma). In yet another embodiment, the control population comprises or alternatively consists essentially of, or yet further consists of samples isolated from a subject who may or may not have a disease (such as a uveal melanoma). In a particular embodiment, the control population comprises or alternatively consists essentially of, or yet further consists of the samples used in the Examples. In a further embodiment, analysis on the samples used in the Examples provide two tertile points as beta values of 0.23 and 0.53 and the three tertile groups are a methylation level having a beta value of less than about 0.23 (low), about 0.23 to about 0.53 (intermediate), and greater than about 0.53 (high). In yet a further embodiment, the two tertile points are beta values of 0.25 an d0.50 and the three tertile groups are a methylation level having a beta value of less than about 0.25 (low), about 0.25 to about 0.50 (intermediate) and greater than about 0.50 (high).

In certain embodiments of these treatment methods as well as other methods and/or embodiments in this disclosure, high methylation levels are detected as (i) a methylation beta value of (a) above about 0.3, (b) above about 0.25, (c) above about 0.75, or (d) above the cutoff for beta value set in Table 2 and/or 7; and/or (ii) an average of all methylation beta values at (a) above about 0.3, (b) above about 0.25, or (c) above about 0.75. Low methylation levels are detected as (i) a methylation beta value of (a) about 0.3 or lower, or (b) about 0.25 or lower, (c) about 0.75 or lower, or (d) the cutoff for beta value set in Table 2 and/or 7 or lower; and/or (ii) as an average of all methylation beta values at (a) about 0.3 or lower, or (b) about 0.25 or lower, or (c) about 0.75 or lower. In one embodiment, the intermediate methylation levels are detected as (i) a methylation beta value from about 0.25 to about 0.75; and/or (ii) an average of all methylation beta values from about 0.25 to about 0.75. Alternatively, a low methylation level further comprises, or alternatively consists essentially of, or yet further consists of a level detected as (i) a methylation beta value from about 0.25 to about 0.75; and/or (ii) an average of all methylation beta values from about 0.25 to about 0.75.

In one aspect, high methylation levels comprise, or alternatively consist essentially of, or yet further consist of a methylation beta value above about 0.3. In a further aspect, high methylation levels comprise, or alternatively consist essentially of, or yet further consist of a methylation beta value above about 0.3, or alternatively above about 0.35, or alternatively above about 0.4, or alternatively above about 0.45, or alternatively above about 0.5, or alternatively above about 0.55, or alternatively above about 0.6, or alternatively above about 0.65 or alternatively above about 0.7, or alternatively above about 0.8, or above about 0.85, or alternatively above about 0.9, or alternatively above about 0.95, or alternatively above about 1, or alternatively above about 1.5, or alternatively above about 2, or alternatively above about 2.5, or alternatively above about 3, or alternatively above about 3.5, or alternatively above about 4, or alternatively above about 4.5, or alternatively above about 5, or alternatively above about 5.5, or alternatively above about 6, or alternatively above about 6.5, or alternatively above about 7, or alternatively above about 8 indicate a decreased probability and/or duration of survival.

In another aspect, low methylation levels comprise, or alternatively consist essentially of, or yet further consist of a methylation beta value of about 0.3 or lower. In a further aspect, low methylation levels comprise, or alternatively consist essentially of, or yet further consist of a methylation beta value of about 0.3 or lower, or alternatively about 0.28 or lower, or alternatively about 0.26 or lower, or alternatively about 0.24 or lower, or alternatively about 0.22 or lower, or alternatively about 0.2 or lower, or alternatively about 0.18 or lower, or alternatively about 0.16 or lower, or alternatively about 0.14 or lower, or alternatively about 0.12 or lower, or alternatively about 0.1 or lower, or alternatively about 0.09 or lower, or alternatively about 0.08 or lower, or alternatively about 0.07 or lower, or alternatively about 0.06 or lower, or alternatively about 0.05 or lower, or alternatively about 0.04 or lower, or alternatively about 0.03 or lower, or alternatively about 0.02 or lower, or alternatively about 0.01 or lower, or alternatively about 0.001 or lower.

Methods of Detection and/or Diagnosis

Further provided herein is a method for detecting high-risk uveal melanoma in a subject comprising, or alternatively consisting essentially of, or yet further consisting of detecting high methylation levels at one or more of: BAP-1, CCNG2 and/or FKBP14 and/or measuring the mRNA expression of JARID2 and TMEM173 and determining ratio of mRNA expression JARID2 to TMEM173 in a sample isolated from the subject. In one aspect, the method further comprises, or alternatively consists essentially of, or yet further consists of detecting high methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7. In another aspect, the method of detection comprises, or alternatively consists essentially of, or yet further consists of detecting high methylation levels at one or more of: BAP-1, CCNG2, FKBP14, PDE4B, SH3D19, SLC6A15 LMCD1, LMCD1-AS1, SENP6, NFIA, CTD-2207023.12, ZNF358, CCDC176, ENTPD5, DTWD1, FAM227B, SESN1, ENPP2 and/or methylation loci identified in Table 2 and/or 7, and/or measuring the mRNA expression of JARID2 and TMEM173 and determining ratio of mRNA expression JARID2 to TMEM173 in a sample isolated from the subject. In a further aspect, the subject is selected when the ratio of mRNA expression JARID2 to TMEM173 is less than about 1. In a particular aspect, the subject is selected when the ratio of mRNA expression JARID2 to TMEM173 is less than about 1, or alternatively less than about 0.95, or alternatively less than about 0.90, or alternatively less than about 0.85, or alternatively less than about 0.80, or alternatively less than about 0.75, or alternatively less than about 0.70, or alternatively less than about 0.65, or alternatively less than about 0.60, or alternatively less than about 0.55, or alternatively less than about 0.50, or alternatively less than about 0.45, or alternatively less than about 0.40, or alternatively less than about 0.35, or alternatively less than about 0.30, or alternatively less than about 0.25, or alternatively less than about 0.20, or alternatively less than about 0.15, or alternatively less than about 0.10, or alternatively less than about 0.05, or alternatively less than about 0.04, or alternatively less than about 0.03, or alternatively less than about 0.02, or alternatively less than about 0.01.

Also provided herein is a method for detecting high-risk uveal melanoma in a subject comprising, or alternatively consisting essentially of, or yet further consisting of detecting high methylation levels at one or more of: BAP-1, CCNG2 and/or FKBP14 and/or a low ratio of mRNA expression JARID2 to TMEM173 in a sample isolated from the subject.

In a further embodiment, the method (for example, the detection step of the method) comprises, or alternatively consists essentially of, or yet further consists of measuring the mRNA expression of JARID2 and TMEM173 and determining a low ratio of mRNA expression JARID2 to TMEM173 in a sample isolated from the subject.

In one embodiment, the method further comprises, or alternatively consists essentially of, or yet further consists of detecting high methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7.

In another embodiment, the method of detection comprises, or alternatively consists essentially of, or yet further consists of detecting high methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or a low ratio of mRNA expression JARID2 to TMEM173 in a sample isolated from the subject.

Additionally or alternatively, the detection is an in vitro and/or ex vivo detection.

In a further embodiment, the method further comprises, or alternatively consists essentially of, or yet further consists of administering to the subject with a high-risk uveal melanoma an effective amount of one or more of: an anti-cancer drug, immunotherapy, chemotherapy, and/or radiation therapy selective for the uveal melanoma (for example, the high-risk melanoma), thereby treating the subject.

Additionally provided herein is a method for detecting low-risk uveal melanoma in a subject comprising, or alternatively consisting essentially of, or yet further consisting of detecting low methylation levels at one or more of: BAP-1, CCNG2 and/or FKBP14 and/or a high ratio of mRNA expression JARID2 to TMEM173 in a sample isolated from the subject.

In a further embodiment, the method (for example, the detection step of the method) comprises, or alternatively consists essentially of, or yet further consists of measuring the mRNA expression of JARID2 and TMEM173 and determining a high ratio of mRNA expression JARID2 to TMEM173 in a sample isolated from the subject.

In one embodiment, the method further comprises, or alternatively consists essentially of, or yet further consists of detecting low methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7.

In another embodiment, the method of detection comprises, or alternatively consists essentially of, or yet further consists of detecting low methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or a high ratio of mRNA expression JARID2 to TMEM173 in a sample isolated from the subject.

Additionally or alternatively, the detection is an in vitro and/or ex vivo detection.

In a further embodiment, the method further comprises, or alternatively consists essentially of, or yet further consists of administering to the subject with a low-risk uveal melanoma an effective amount of one or more of: an anti-cancer drug, immunotherapy, chemotherapy, and/or radiation therapy selective for the uveal melanoma (for example, the low-risk melanoma), thereby treating the subject.

Further provided herein is a method for detecting an intermediate-risk uveal melanoma in a subject comprising, or alternatively consisting essentially of, or yet further consisting of detecting intermediate methylation levels at one or more of: BAP-1, CCNG2 and/or FKBP14 and/or an intermediate ratio of mRNA expression JARID2 to TMEM173 in a sample isolated from the subject.

In a further embodiment, the method (for example, the detection step of the method) comprises, or alternatively consists essentially of, or yet further consists of measuring the mRNA expression of JARID2 and TMEM173 and determining an intermediate ratio of mRNA expression JARID2 to TMEM173 in a sample isolated from the subject.

In one embodiment, the method further comprises, or alternatively consists essentially of, or yet further consists of detecting intermediate methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7.

In another embodiment, the method of detection comprises, or alternatively consists essentially of, or yet further consists of detecting intermediate methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or an intermediate ratio of mRNA expression JARID2 to TMEM173 in a sample isolated from the subject.

Additionally or alternatively, the detection is an in vitro and/or ex vivo detection.

In a further embodiment, the method further comprises, or alternatively consists essentially of, or yet further consists of administering to the subject with an intermediate-risk uveal melanoma an effective amount of one or more of: an anti-cancer drug, immunotherapy, chemotherapy, and/or radiation therapy selective for the uveal melanoma (for example, the intermediate-risk melanoma), thereby treating the subject.

In one embodiment, the BAP-1 methylation levels in the methods described herein are detected at the methylation loci identified in Tables 2, 3, 4 and/or 5. In one particular embodiment, BAP-1 methylation levels in the methods described herein are detected at the methylation locus CGI:chr3:52409661-52410118.

### Methods of Prognosis and Others

This disclosure also provides methods of determining one or more of the following: (1) prognosis of a subject having uveal melanoma (UM), (2) an probability of having a high-risk UM with poor prognosis, (3) a probability of having a UM tumor with partial or full monosomy 3, or (4) a possibility of having a BAP1 mutation in a UM comprising, or alternatively consisting essentially of, or yet further consisting of detecting methylation levels at one or more of: BAP-1, CCNG2 and/or FKBP14 and/or measuring the mRNA expression of JARID2 and TMEM173 and determining ratio of mRNA expression JARID2 to TMEM173 in a sample isolated from the subject, wherein high methylation levels and/or the ratio of mRNA expression JARID2 to TMEM173 of less than about 1 indicate a decreased probability and/or duration of survival and low methylation levels and/or the ratio of mRNA expression JARID2 to TMEM173 of greater than about 1 indicate an increased probability and/or duration of survival. In one aspect, the method further comprises, or alternatively consists essentially of, or yet further consists of detecting methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, wherein high methylation levels indicate a decreased probability and/or duration of survival and low methylation levels indicates an increased probability and/or duration of survival. In another aspect, the method of detection comprises, or alternatively consists essentially of, or yet further consists of detecting high methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or measuring the mRNA expression of JARID2 and TMEM173 and determining ratio of mRNA expression JARID2 to TMEM173 in a sample isolated from the subject, wherein high methylation levels and/or the ratio of mRNA expression JARID2 to TMEM173 of less than about 1 indicate a decreased probability and/or duration of survival and low methylation levels and/or the ratio of mRNA expression JARID2 to TMEM173 of greater than about 1 indicate an increased probability and/or duration of survival.

In one embodiment, the detection and/or measurement is performed in vitro and/or ex vivo.

In one aspect the ratio of mRNA expression JARID2 to TMEM173 of less than about 1, or alternatively less than about 0.95, or alternatively less than about 0.90, or alternatively less than about 0.85, or alternatively less than about 0.80, or alternatively less than about 0.75, or alternatively less than about 0.70, or alternatively less than about 0.65, or alternatively less than about 0.60, or alternatively less than about 0.55, or alternatively less than about 0.50, or alternatively less than about 0.45, or alternatively less than about 0.40, or alternatively less than about 0.35, or alternatively less than about 0.30, or alternatively less than about 0.25, or alternatively less than about 0.20, or alternatively less than about 0.15, or alternatively less than about 0.10, or alternatively less than about 0.05, or alternatively less than about 0.04, or alternatively less than about 0.03, or alternatively less than about 0.02, or alternatively less than about 0.01 indicate a decreased probability and/or duration of survival.

In another aspect the ratio of mRNA expression JARID2 to TMEM173 is greater than about 1, or alternatively greater than about 1.1, or alternatively greater than about 1.2, or alternatively greater than about 1.3, or alternatively greater than about 1.4, or alternatively greater than about 1.5, or alternatively greater than about 1.6, or alternatively greater than about 1.7, or alternatively greater than about 1.8, or alternatively greater than about 1.9, or alternatively greater than about 2, or alternatively greater than about 2.1, or alternatively greater than about 2.2, or alternatively greater than about 2.3, or alternatively greater than about 2.4, or alternatively greater than about 2.5, or alternatively greater than about 2.6, or alternatively greater than about 2.7, or alternatively greater than about 2.8, or alternatively greater than about 2.9, or alternatively greater than about 3, or alternatively greater than 3.1, or alternatively greater than about 3.2, or alternatively greater than about 3.5, or alternatively greater than about 4, or alternatively greater than about 4.5, or alternatively greater than about 5 indicate a decreased probability and/or duration of survival.

In one embodiment, the method further comprises administering to a subject an effective amount of one or more of: an anti-cancer drug, immunotherapy, chemotherapy, and/or radiation therapy suitable for the determined prognosis of a subject, thereby treating the subject.

This disclosure also provides methods of determining prognosis of a subject having uveal melanoma comprising, or alternatively consisting essentially of, or yet further consisting of detecting methylation levels at one or more of: BAP-1, CCNG2 and/or FKBP14 and/or determining ratio of mRNA expression JARID2 to TMEM173 in a sample isolated from the subject. In one embodiment, the detection and/or measurement is performed in vitro and/or ex vivo.

High methylation levels and/or a low ratio of mRNA expression JARID2 to TMEM173 indicate a less favorable prognosis and low methylation levels and/or a high ratio of mRNA expression JARID2 to TMEM173 indicate a more favorable prognosis.

As used herein, a less favorable prognosis comprises, or alternatively consists essentially of, or yet further consists of a decreased overall survival, and/or an increased probability of having a metastasis (for example in liver) while more favorable prognosis comprises, or alternatively consists essentially of, or yet further consists of an increased overall survival and/or a decreased probability of having a metastasis (such as in liver).

In certain embodiments, an intermediate methylation level and/or an intermediate ratio of mRNA expression JARID2 to TMEM173 indicate an intermediate prognosis which comprises an intermediate overall survival, and/or an intermediate probability of having a metastasis (such as in liver).

In a further embodiment, the method (for example, the determination step of the method) comprises, or alternatively consists essentially of, or yet further consists of measuring the mRNA expression of JARID2 and TMEM173 and determining a ratio of mRNA expression JARID2 to TMEM173 in a sample isolated from the subject.

In one embodiment, the method further comprises, or alternatively consists essentially of, or yet further consists of detecting methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, wherein high methylation levels indicate a less favorable prognosis and low methylation levels indicate more favorable prognosis.

In another embodiment, the method (for example, the method step of detection and/or determination) comprises, or alternatively consists essentially of, or yet further consists of detecting methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7, and/or determining ratio of mRNA expression JARID2 to TMEM173 in a sample isolated from the subject.

The high methylation levels and/or the low ratio of mRNA expression JARID2 to TMEM173 indicate a less favorable prognosis. Additionally or alternatively, the high methylation levels and/or the low ratio of mRNA expression JARID2 to TMEM173 indicate one or more of the following: (1) a decreased probability and/or duration of survival, (2) an increased probability of having a metastasis, (3) an increased probability of having a high-risk UM with poor prognosis, (4) an increased probability of having a UM tumor with partial or full monosomy 3, or (5) an increased possibility of having a BAP1 mutation.

The low methylation levels and/or the high ratio of mRNA expression JARID2 to TMEM173 indicate more favorable prognosis. Additionally or alternatively, the low methylation levels and/or the high ratio of mRNA expression JARID2 to TMEM173 indicate one or more of the following: (1) an increased probability and/or duration of survival, (2) a decreased probability of having a metastasis, (3) an increased probability of having a low-risk UM with good prognosis, (4) an increased probability of having a UM tumor with disomy 3, or (5) an increased possibility of having a wildtype BAP1.

Also, the intermediate methylation levels and/or the intermediate ratio of mRNA expression JARID2 to TMEM173 indicate intermediate favorable prognosis. Additionally or alternatively, the intermediate methylation levels and/or the intermediate ratio of mRNA expression JARID2 to TMEM173 indicate one or more of the following: 1) an intermediate probability and/or duration of survival, (2) an intermediate probability of having a metastasis, (3) an intermediate probability of having a low-risk UM with good prognosis, (4) an intermediate probability of having a high-risk UM with poor prognosis, (5) an intermediate probability of having a UM tumor with disomy 3 or partial/full monosomy 3, or (6) an intermediate possibility of having a wildtype BAP1 or a BAP1 mutation.

In a further embodiment, the method (for example, the detection and/or determination step of the method) comprises, or alternatively consists essentially of, or yet further consists of measuring the mRNA expression of JARID2 and TMEM173 and determining a ratio of mRNA expression JARID2 to TMEM173 in a sample isolated from the subject.

In yet a further embodiment, the method further comprises, or alternatively consists essentially of, or yet further consists of administering to the subject an effective amount of one or more of: an anti-cancer drug, immunotherapy, chemotherapy, and/or radiation therapy suitable for the determined prognosis of a subject, thereby treating the subject. The detection and/or measurement may be performed in vitro and/or ex vivo.

Further provided is a method comprising quantifying a methylation level of a loci in a sample isolated from a subject. The loci comprises one or more of the loci identified in Table 2 and/or 7 and/or the loci within one or more of the following genes: BAP-1, CCNG2 and/or FKBP14, PDE4B, SH3D19, SLC6A15 LMCD1, LMCD1-AS1, SENP6, NFIA, CTD-2207023.12, ZNF358, CCDC176, ENTPD5, DTWD1, FAM227B, SESN1, or ENPP2. Optionally, a DNA comprising the one or more loci have been enriched and/or amplified prior to or concurrently with the quantification step.

In one embodiment, the method further comprises obtaining a sample detected with a methylation level at the one or more loci, and/or an average methylation level at more than one loci, as having a beta value of (a) about 0.3 or lower, (b) greater than about 0.3, (c) about 0.25 or lower, (d) about 0.25 to about 0.50, (e) about 0.25 to about 0.75, (f) greater than about 0.25, (g) about 0.50 or lower, (h) greater than about 0.50, (i) about 0.75 or lower, (j) greater than about 0.75, (k) about 0.23 or lower, (1) about 0.23 to about 0.53, (m) about 0.53 or lower, (n) greater than about 0.53, (o) about the cutoff for the beta value set in Table 2 and/or 7 or lower, or (p) greater than the cutoff for the beta value set in Table 2 and/or 7. In another embodiment, the method further comprises obtaining a sample detected with any methylation level as disclosed herein, for example, under the section titled Methylation Levels.

In certain embodiments, a methylation level at the one or more loci, and/or an average methylation level at more than one loci, having a beta value of (a) about 0.3 or lower, (c) about 0.25 or lower, (g) about 0.50 or lower, (i) about 0.75 or lower, (k) about 0.23 or lower, (m) about 0.53 or lower, or (o) about the cutoff for the beta value set in Table 2 or lower indicates one or more of the following: (1) an increased probability and/or duration of survival, (2) a decreased probability of having a metastasis, (3) an increased probability of having a low-risk UM with good prognosis, (4) an increased probability of having a UM tumor with disomy 3, or (5) an increased possibility of having a wildtype BAP1.

In certain embodiments, a methylation level at the one or more loci, and/or an average methylation level at more than one loci, having a beta value of (d) about 0.25 to about 0.50, (e) about 0.25 to about 0.75, or (1) about 0.23 to about 0.53, indicates one or more of the following: (1) an intermediate probability and/or duration of survival, (2) an intermediate probability of having a metastasis, (3) an intermediate probability of having a low-risk UM with good prognosis, (4) an intermediate probability of having a high-risk UM with poor prognosis, (5) an intermediate probability of having a UM tumor with disomy 3 or partial/full monosomy 3, or (6) an intermediate possibility of having a wildtype BAP1 or a BAP1 mutation.

In certain embodiments, a methylation level at the one or more loci, and/or an average methylation level at more than one loci, having a beta value of (b) greater than 0.3, (f) greater than about 0.25, (h) greater than about 0.50, (j) greater than about 0.75, (n) greater than about 0.53 or (p) greater than the cutoff for the beta value set in Table 2 indicates one or more of the following: (1) a decreased probability and/or duration of survival, (2) an increased probability of having a metastasis, (3) an increased probability of having a high-risk UM with poor prognosis, (4) an increased probability of having a UM tumor with partial or full monosomy 3, or (5) an increased possibility of having a BAP1 mutation.

In certain embodiments, the quantification step comprises treating the genomic DNA with bisulfite and converting unmethylated cytosines to uracil. Additionally or alternatively, the quantification step comprises binding an antibody or a fragment thereof specifically to a methylated loci.

Additionally or alternatively, the method further comprises quantifying one or more of the following in the isolated sample: (a) mRNA level of JARID2 and mRNA level of TMEM173; (b) ratio of mRNA expression JARID2 to TMEM173; or (c) ratio of mRNA expression TMEM173 to JARID2.

Additionally provided is a method comprising quantifying one or more of the following in a sample isolated from a subject: (a) mRNA level of JARID2 and mRNA level of TMEM173; (b) ratio of mRNA expression JARID2 to TMEM173; or (c) ratio of mRNA expression TMEM173 to JARID2. Optionally mRNAs of JARID2 and TMEM173 have been enriched and/or reverse-transcribed prior to or concurrently with the quantification.

In one embodiments, the method further comprises obtaining mRNAs of JARID2 and TMEM173 having a ratio of mRNA expression JARID2 to TMEM173 at (a) less than about 0.3, (b) about 0.3 to about 1, (c) about 0.3 to about 2, (d) more than 0.3, (e) less than about 1, (f) about 1 to about 2, (g) more than about 1, (h) less than about 2, or (i) more than about 2. In another embodiment, the method further comprises obtaining mRNAs of JARID2 and TMEM173 having any ratio of mRNA expression JARID2 to TMEM173 as disclosed herein, for example, under the section titled Ratio of mRNA Expression JARID2 to TMEM173.

In certain embodiments, a ratio of mRNA expression JARID2 to TMEM173 or an equivalent thereof at (a) less than about 0.3, (e) less than about 1, or (h) less than about 2 indicates one or more of the following: (1) a decreased probability and/or duration of survival, (2) an increased probability of having a metastasis, (3) an increased probability of having a high-risk UM with poor prognosis, (4) an increased probability of having a UM tumor with partial or full monosomy 3, or (5) an increased possibility of having a BAP1 mutation.

In certain embodiments, a ratio of mRNA expression JARID2 to TMEM173 or an equivalent thereof at (b) about 0.3 to about 1, (c) about 0.3 to about 2, or (f) about 1 to about 2, indicates one or more of the following: (1) an intermediate probability and/or duration of survival, (2) an intermediate probability of having a metastasis, (3) an intermediate probability of having a low-risk UM with good prognosis, (4) an intermediate probability of having a high-risk UM with poor prognosis, (5) an intermediate probability of having a UM tumor with disomy 3 or partial/full monosomy 3, or (6) an intermediate possibility of having a wildtype BAP1 or a BAP1 mutation.

In certain embodiments, a ratio of mRNA expression JARID2 to TMEM173 or an equivalent thereof at (d) more than 0.3, (g) more than about 1, or (i) more than about 2 indicates (1) an increased probability and/or duration of survival, (2) a decreased probability of having a metastasis, (3) an increased probability of having a low-risk UM with good prognosis, (4) an increased probability of having a UM tumor with disomy 3, or (5) an increased possibility of having a wildtype BAP1.

Non-limiting examples of increased probability and/or duration of survival include increased overall survival (OS), increased progression free survival (PFS), increased disease free survival (DFS), increased time to tumor recurrence (TTR) and increased time to tumor progression (TTP). Non-limiting examples of decreased probability and/or duration of survival include decreased overall survival (OS), decreased progression free survival (PFS), decreased disease free survival (DFS), decreased time to tumor recurrence (TTR) and decreased time to tumor progression (TTP).

In one embodiment, the BAP-1 methylation levels in the methods described herein are detected at the methylation loci identified in Tables 2, 3, 4 and/or 5. In one particular embodiment, BAP-1 methylation levels in the methods described herein are detected at the methylation locus CGI:chr3:52409661-52410118. In one embodiment, the quantification is performed in vitro and/or ex vivo.

The sample isolated from the subject is one or more of a blood, a urine, an aqueous vitreous humor, a tumor biopsy and/or a liquid biopsy sample. Biopsy specimens from fine needle aspiration or enucleation, paraffin-embedded or frozen specimens, as well as cell-free DNA from patients with uveal melanoma can also be used as samples.

In one aspect, the subject on whom the methods of this disclosure are carried out is a mammal. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a Uveal melanoma.

### Kits

Also described herein is a kit comprising, or alternatively consisting essentially of, or yet further consisting of probes primers, optional bisulfate salt or solution, and optional antibodies for carrying out the methods of this disclosure, and optional instructions for use. In a further aspect, the instruction for use provide directions to conduct any of the methods disclosed herein.

The kit components, (e.g., reagents) can be packaged in a suitable container. The kit can also comprise, or alternatively consist essentially of, or yet further consist of, e.g., a carrier such as a buffering agent, a preservative or a protein-stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### EXPERIMENTAL

### Example 1

Uveal melanoma (UM) is relatively uncommon, but when metastatic is a deadly disease. Applicants have found that a high methylation level on the gene BAP1 at a single locus (CGI coordinates, CGI:chr3:52409661-52410118) can predict patient survival and can be used to estimate prognostic classes. BRCA1-associated protein-1 (BAP1), located at 3p21, encodes a deubiquitinating enzyme involved in the removal of ubiquitin from proteins. Alteration on BAP1 mRNA expression or mutations on the BAP1 gene have been associated with UM metastasis since it naturally shows tumor suppressor activity. On another note, Applicants have also found that an assay that evaluates the relative expression of two genes from bulk sequencing and identified a subpopulation of cells that accounts for poor survival outcomes by looking at the ratio of mRNA expression of two genes JARID2 and TMEM1173.

When evaluating this BAP1 methylation site, if a sample had a beta value of >0.3, the patient had a 50% chance of survival at 5 years (positive predictive value (PPV)=0.5). If on the other hand the beta value was <0.3, the chance of survival was 95% (negative predictive value (NPV)=0.95). The test has 91% sensitivity and 63% specificity based on the 80 patients' samples analyzed so far. The specificity is low because mortality from metastatic uveal melanoma can occur at more than 5 years and the scientists used a 5 years' cut-off. The type of samples that can be used for this test are: biopsy specimens from fine needle aspiration or enucleation, paraffin-embedded or frozen specimens, as well as cell-free DNA from patients with Uveal melanoma.

TABLE 1

| | | Deceased | Alive | Sensitivity | Specificity | PPV | NPV |
|---|---|---|---|---|---|---|---|
| BAP1 | High | 21 | 21 | 0.91304348 | 0.63157895 | 0.5 | 0.94736842 |
| | Low | 2 | 36 | | | | |
| PDE4B | High | 21 | 21 | 0.91304348 | 0.63157895 | 0.5 | 0.94736842 |
| | Low | 2 | 36 | | | | |
| SH3D19 | High | 22 | 28 | 0.95652174 | 0.50877193 | 0.44 | 0.96666667 |
| | Low | 1 | 29 | | | | |
| CCNG2 | High | 21 | 18 | 0.91304348 | 0.68421053 | 0.53846154 | 0.95121951 |
| | Low | 2 | 39 | | | | |
| FKBP14 | High | 22 | 19 | 0.95652174 | 0.66666667 | 0.53658537 | 0.97435897 |
| | Low | 1 | 38 | | | | |
| SLC6A15 | High | 21 | 19 | 0.91304348 | 0.66666667 | 0.525 | 0.95 |
| | Low | 2 | 38 | | | | |

Applicants analyzed bulk sequencing data from uveal melanoma which are publicly available through the Cancer Genome Atlas (TCGA) and have found that intratumor heterogeneity can be inferred from bulk sequencing by analyzing the ratio of gene expression of a set of genes. Applicants have also found that high BAP1 methylation at a single locus can predict survival and could be used to estimate the prognostic classes. Applicants demonstrate using a published cohort that a geneset or methylation data of a single gene can accurately predict survival in patients with primary uveal melanoma. Survival estimates based on gene expression (mRNA) of two genes and methylation patterns of BAP1 gene. The current clinical testing used in prognostication for uveal melanoma is DecisionDx-UM is offered by castle biosciences. It relies on a 12-gene panel which was derived from bulk sequencing. Applicants believe that their data inferred from single cell identities can reflect the percentage of two cellular state in uveal melanoma and is non-inferior to the castle testing. In addition, the methylation data from BAP1 is highly predictive of survival and would be performed at a much lower cost and faster turnaround time compared to the gene panel testing. Sample handling would also be easier as DNA is much more stable than RNA. In addition, it could serve as a basis for development of blood levels.

Specifically, Applicants have found that methylation of BAP1 at a single location, North-Shore of CpG island (CGI coordinates, CGI:chr3:52409661-52410118), (Genomic coordinates 52408017-8) reflects BAP1 genomic copy number and mutational status with a high confidence. Applicants found that a methylation beta value less than 0.3 is a strong predictor of survival. Current prognostication markers include karyotypic analysis, FISH, Gene expression profiling and histological assessment of cellular morphology. BAP1 methylation is non-inferior to all these methods, and superior to most. The big advantage is that testing methylation at a single locus would cost less than $100. The current gene expression-profiling test offered by Castle Biosciences costs more than $1,500.00. When applied to an independent cohort through the Cancer Genome Atlas (n=80), the BAP1 methylation value had a sensitivity of 91.3%. If a sample had a beta value of >0.3, the patient had a 50% chance of survival at 5 years (Positive predictive value 0.5). If on the other hand the beta value was <0.3, the chance of survival was 95% (negative predictive value 0.95). Another test which could supplement this is by assaying a relative expression of two genes from the bulk sequencing. Applicants found that there is a subpopulation of cells that accounts for poor survival outcomes. Applicants looked at a ratio of mRNA expression of two genes JARID2 and TMEM173. We divided samples into two categories (JARID2/TMEM173 (J/T) >1 and JARID2/TMEM173 (J/T)<1). There were 23 patients in the first category (J/T ratio >1) and 55 samples in the second category (J/T ratio <1). Survival at 5 years was 100% for J/T ratio >1 and 18% for J/T ratio <1, see Kaplan Meier attached. This ratio can be used to other gene sets, which we identified as well.

Using the tier 1 or top 7-12 methylation markers, this test can be used in blood, urine and/or aqueous vitreous humor in addition to the biopsies of the eye tumor. Using blood or urine would be much less invasive test for the patient and with less negative side effects. This test can be applied to biopsy specimens from fine needle aspiration or enucleation, paraffin-embedded or frozen specimens. It can also be applied to cell-free DNA from patients with Uveal melanoma. Methylations sites on BAP1 is used as a surrogate marker for detecting low copy number and mutations in BAP1 which would need deep sequencing (which is expensive), while this test based on methylation, would be cheaper and easier to do. Applicants identified single genomic sites in uveal melanoma, the most common primary intraocular tumor, where methylation status can predict
a) overall survival,
b) the status of BAP1 copy number or chromosome 3 genomic copy number status and
c) the mutational status of BAP1.

This methylation signature can be applied on biopsies from uveal melanoma specimens, enucleation uveal melanoma specimens, frozen or paraffin-embedded tissue, as well as blood, urine, aqueous or vitreous humor. Identifying patients with ‘high-risk’ tumors is useful for many reasons:
a) Provides Prognostic information to patients.
b) Patients with high-risk tumors should undergo surveillance at shorter intervals (i.e. Liver CT scans or MRI and PET Scan), since earlier identification of metastasis can lead to improved survival.
c) There is currently no effective treatment for metastatic disease. There are few clinical trials for adjuvant therapies or therapies aimed at managing the primary and metastatic disease. Identifying patients with the high-risk tumor can help in enrolling patients in clinical trials and identifying the role of these therapies in different tumor sub sets.
d) Identifying the methylation signature from liquid biopsies avoids the complications of intraocular biopsies.
e) Identifying the methylation signature from liquid biopsies has the potential to diagnose metastatic disease or recurrence.

The current clinical testing used in prognostication for uveal melanoma is DecisionDx-UM is offered by Castle Biosciences (http://castlebiosciences.com). It relies on mRNA expression from a 12-gene panel which was derived from bulk sequencing. Beside Castle Biosciences, Impactgenetics (http://impactgenetics.com) offers a genetic prognostic test for uveal melanoma. This test relies on multiplex ligation-dependent probe amplification (MLPA) on chromosomes 1, 3, 6 and 8 to detect genomic copy number variations, microsatellite analysis (MSA) on chromosome 3 to detect chromosome copy loss and/or isodisomy and sequencing of GNAQ, GNA11, SF 3B1 and EIF1AX to detect frequently occurring mutations in UM tumors. They do not offer BAP1 sequencing since it requires very deep sequencing. Since this test relies on isolated DNA, the proposed technology (methylation analysis) can easily be applied to their current panel and replace many of these tests.

Genetic prognostication of uveal melanoma is offered to the majority of patients in the United States. However, the cost of these tests prevent their use on a larger scale (i.e. worldwide). The cost of DecisionDx (Cash value for uninsured patients) is $1500 and Impactgenetics test is $950. Methylation sequencing is much cheaper. Additionally, shipping specimens on dry ice from overseas is another obstacle to using the gene expression profile test. The Applicant's disclosure has the following advantages:
1. Low cost—Measuring level of methylation at a single genomic locus is cheaper than analyzing RNA, genomic copy number alterations or deep sequencing of BAP1.
2. Reproducible—Measuring level of methylation at a single genomic locus is very reproducible.
   Routine exome sequencing of BAP1 can miss mutations in the gene, many of which are intronic. Deep sequencing on the other hand is expensive. By measuring methylation data at a single locus Applicants can overcome this obstacle.
3. Easier to handle specimens—The biopsies from the primary tumor can be obtained and shipped at room temperature. The current clinical testing relies on analysis of mRNA which is more tedious to handle (requires freezing at −80 and shipping on dry ice).
4. Allows obtaining biopsies in a solution using a vitrectomy machine—Biopsies of the primary tumor (intraocular) can be done with a fine needle or a vitrectomy machine. The advantages of using a vitrectomy machine is to increase the yield of the tumor which also avoids sampling error. However, since the vitrectomy probe is primed with a saline solution, the sample is diluted and consequently affects the stability of mRNA. This prevents the use of this technique in obtaining specimens for gene expression profile since it leads to de-stabilization of the mRNA. The biopsy specimen can be obtained in a diluted form prior to performing the methylation sequencing.
5. Liquid Biopsies—The methylation signature can be applied on biopsies from the primary tumor. It can also be reflected in the blood, urine and vitreous or aqueous humor. This has the potential to reduce or eliminate the need for obtaining a biopsy of the tumor. Intraocular biopsies have associated side effects which include eye infection, bleeding, retinal detachment, tumor spread to adjacent structures, Hypotony, glaucoma, loss of vision and cataract formation.

### Example 2: Identification of Methylation Sites

Applicants analyzed the methylation signature obtained from 80 uveal specimens. These underwent whole exome sequencing, RNA sequencing and have associated clinical findings including survival. This allowed us to isolate a set of genomic loci where methylation is highly associated with poor survival and can reflect BAP1 and chromosome 3 copy number status and BAP1 mutations. Each of these loci can independently predict these outcomes at a high confidence. The decision to pick more than one locus is to increase the test sensitivity when applied to liquid biopsies.

Beta value is the average of methylated loci over non-methylated. i.e. a beta value of 0.1 means 10% of a specific genomic locus is methylated. As shown below, the methylation value at each locus can predict survival or BAP1 status with a high precision. There is no need to include a panel.

TABLE 2
Methylation Loci and Guideline for Beta-Value Cutoffs

| Unique | Chromo- | | | Cutoff for | |
|---|---|---|---|---|---|
| identifier | some | Start Site | Associated Genes | Beta Value | Tier |
| 130560 | chr3 | 8503226 | LMCD1 | 0.35 | 1 |
| | | | LMCD1-AS1 | | |
| 75967 | chr6 | 75600835 | SENP6 | 0.7 | 1 |
| 111951 | chr2 | 219595792 | | 0.5 | 1 |
| 382471 | chr4 | 77155998 | CCNG2 | 0.4 | 1 |
| 343359 | chr1 | 61084310 | NFIA | 0.5 | 1 |
| 309887 | chr3 | 52408017 | BAP1 | 0.3 | 1 |
| 41573 | chr19 | 7515331 | CTD-2207O23.12 | 0.5 | 1 |
| | | | ZNF358 | | |
| 7446 | chr19 | 7515389 | CTD-2207O23.12 | 0.6 | 1 |
| | | | ZNF358 | | |
| 338430 | chr14 | 74019948 | CCDC176 | 0.2 | 2 |
| | | | ENTPD5 | | |
| 89586 | chr15 | 49621399 | DTWD1 | 0.3 | 2 |
| | | | FAM227B | | |
| 406409 | chr1 | 66333736 | PDE4B | 0.4 | 2 |
| 161744 | chr6 | 109010239 | SESN1 | 0.5 | 2 |
| 267855 | chr8 | 119639412 | ENPP2 | 0.5 | 2 |

These are the statistics for the use of each methylation locus in predicting BAP1 genomic copy number, also a surrogate for chromosome 3 copy number. BAP1 copy loss was defined as a less than a relative value of 0.2. This is equivalent to monosomy 3 in 20% of the cells. The positive predictive value ranges from 98% to 100%. The negative predictive value ranges from 90 to 97%.

TABLE 3

| | | | Positive | Negative |
|---|---|---|---|---|
| Unique | | | predictive | Predictive |
| identifier | Sensitivity | Specificity | value | Value |
| 130560 | 0.98 | 1.00 | 1.00 | 0.97 |
| 75967 | 0.95 | 1.00 | 1.00 | 0.95 |
| 111951 | 0.93 | 1.00 | 1.00 | 0.93 |
| 382471 | 0.91 | 1.00 | 1.00 | 0.90 |
| 343359 | 0.93 | 1.00 | 1.00 | 0.93 |
| 309887 | 0.95 | 0.97 | 0.98 | 0.95 |
| 41573 | 0.95 | 0.97 | 0.98 | 0.95 |
| 7446 | 0.95 | 0.97 | 0.98 | 0.95 |
| 338430 | 0.93 | 1.00 | 1.00 | 0.93 |
| 89586 | 0.91 | 1.00 | 1.00 | 0.90 |
| 406409 | 0.95 | 0.97 | 0.98 | 0.95 |
| 161744 | 0.93 | 1.00 | 1.00 | 0.93 |
| 267855 | 0.91 | 0.97 | 0.98 | 0.90 |

These are the statistics for the use of each methylation locus in predicting BAP1 mutations. The positive predictive value ranges from 60% to 67%. Since it is hard to identify all mutations in BAP1, the positive predictive value is low. i.e. if Applicants sequence the specimens with high methylation value Applicants may be able to detect more BAP1 mutations than what has been identified. The negative predictive values range from 97% to 100%.

TABLE 4

| | | | Positive | Negative |
|---|---|---|---|---|
| Unique | | | predictive | Predictive |
| identifier | Sensitivity | Specificity | value | Value |
| 130560 | 1.00 | 0.70 | 0.62 | 1.00 |
| 75967 | 1.00 | 0.72 | 0.63 | 1.00 |
| 111951 | 1.00 | 0.74 | 0.65 | 1.00 |
| 382471 | 1.00 | 0.76 | 0.67 | 1.00 |
| 343359 | 1.00 | 0.74 | 0.65 | 1.00 |
| 309887 | 0.96 | 0.69 | 0.60 | 0.97 |
| 41573 | 1.00 | 0.70 | 0.62 | 1.00 |
| 7446 | 1.00 | 0.70 | 0.62 | 1.00 |
| 338430 | 1.00 | 0.74 | 0.65 | 1.00 |
| 89586 | 1.00 | 0.76 | 0.67 | 1.00 |
| 406409 | 1.00 | 0.70 | 0.62 | 1.00 |
| 161744 | 1.00 | 0.74 | 0.65 | 1.00 |
| 267855 | 1.00 | 0.74 | 0.65 | 1.00 |

These are the statistics for the use of each methylation locus in predicting overall survival. The positive predictive value ranges from 50% to 54%. Patients suffering from uveal melanoma can have late metastases. Applicants expect that the positive predictive value will increase if survival is measured at a longer follow up interval. The negative predictive values range from 92% to 95%. One of the patients in this cohort died from a different cause, unrelated to uveal melanoma. Since Applicants are reporting overall survival Applicants did not exclude that patient.

TABLE 5

| | | | Positive | Negative |
|---|---|---|---|---|
| Unique | | | predictive | Predictive |
| identifier | Sensitivity | Specificity | value | Value |
| 130560 | 0.91 | 0.63 | 0.50 | 0.95 |
| 75967 | 0.91 | 0.65 | 0.51 | 0.95 |
| 111951 | 0.91 | 0.67 | 0.53 | 0.95 |
| 382471 | 0.91 | 0.68 | 0.54 | 0.95 |
| 343359 | 0.91 | 0.67 | 0.53 | 0.95 |
| 309887 | 0.91 | 0.63 | 0.50 | 0.95 |
| 41573 | 0.91 | 0.63 | 0.50 | 0.95 |
| 7446 | 0.88 | 0.63 | 0.50 | 0.92 |
| 338430 | 0.91 | 0.67 | 0.53 | 0.95 |
| 89586 | 0.91 | 0.68 | 0.54 | 0.95 |
| 406409 | 0.91 | 0.63 | 0.50 | 0.95 |
| 161744 | 0.91 | 0.67 | 0.53 | 0.95 |
| 267855 | 0.91 | 0.67 | 0.53 | 0.95 |

TABLE 6
5-year survival rate of patients suffering from uveal
melanoma with high or low methylation levels at
chromosomal site with the unique identifier: 309887

| 5-yr survival | Dead | Alive |
|---|---|---|
| High Methylation | 21 | 21 |
| Low Methylation | 2 | 36 |

Sensitivity 91%
Specificity 63%
Negative predictive value 0.95
Positive predictive value 0.5

### Example 3

Highly metastatic primary UM tumors differ from their more indolent counterparts in that (1) they tend to have an “epithelioid” morphology with enlarged nuclei; (2) they are often monosomic for chromosome 3; (3) they frequently harbor mutations in the BAP1 gene (located on chromosome 3); and (4) they exhibit a distinctive gene expression signature.

Applicant's research has identified significant intratumor heterogeneity whereby each tumor contained a heterogeneous admixture of cells that resembled both phenotypes (high risk and low risk) to varying degrees. Further, Applicant identified high-risk UM with good, intermediate and poor prognosis; identified tumors with partial or full monosomy 3, as well as disomy 3, identified tumors with pathogenic mutations in BAP1, as well as those with wild type BAP1 gene, identified the preponderance of tumor cells with high-risk features in UM, predicted overall survival of patients with UM (primary and/or metastatic), and predicted the chance of metastasis, usually to the liver, lungs and/or distant organs. These markers comprises one or more of the following:
(1) high relative DNA methylation of the following genes especially at the loci identified in Table 7: (a) BAP1, and (b) In addition to LMCD1 LMCD1-AS1, SENP6, CCNG2, NFIA, CTD-2207O23.12/ZNF358, CTD-2207O23.12/ZNF358, CCDC176/ENTPD5, DTWD1/FAM227B, PDE4B, SESN1 and ENPP2;
(2) mRNA expression of JARID2;
(3) mRNA expression of TMEM173 (STING); or
(4) Ratio of expression of JARID2 relative to TMEM173 (STING) or TMEM173 (STING) relative to JARID2.

These markers were/are applied on: specimens from UM (primary or metastatic); tumor tissue in freshly obtained specimens; tumor tissue in paraffin embedded tissue; frozen tumor tissue; tumor tissue in fixative; and cell free DNA and or circulating tumor DNA in biological fluids including whole blood, plasma, serum, urine, aqueous humor and vitreous humor.

TABLE 7
Methylation Loci

| | | | Composite Element | | | |
|---|---|---|---|---|---|---|
| | | | reference methylation | | | |
| ID | Chromosome | Gene_Symbol | array 450 | Start | CGI_Coordinate | Feature_Type |
| 309887 | chr3 | BAP1 | cg16871520 | 52408017 | CGI: chr3: | N_Shore |
| | | | | | 52409661-52410118 | |
| 7446 | chr19 | CTD-2207O23.12; | cg00351537 | 7515389 | CGI: chr19: | N_Shore |
| | | ZNF358 | | | 7515938-7516529 | |
| 41573 | chr19 | CTD-2207O23.12; | cg02026535 | 7515331 | CGI: chr19: | N_Shore |
| | | ZNF358 | | | 7515938-7516529 | |
| 75967 | chr6 | SENP6 | cg03760308 | 75600835 | CGI: chr6: | N_Shore |
| | | | | | 75601614-75602847 | |
| 89586 | chr15 | DTWD1: | cg04468564 | 49621399 | CGI: chr15: | S_Shore |
| | | FAM227B | | | 49620788-49621285 | |
| 111951 | chr2 | | cg05656360 | 219595792 | CGI: chr2: | N_Shore |
| | | | | | 219597585-219598222 | |
| 130560 | chr3 | LMCD1; | cg06697448 | 8503226 | CGI: chr3: | S_Shore |
| | | LMCD1-AS1 | | | 8501515-8501893 | |
| 161744 | chr6 | SESN1 | cg08336300 | 109010239 | CGI: chr6: | S_Shore |
| | | | | | 109009082-109009834 | |
| 267855 | chr8 | ENPP2 | cg14409958 | 119639412 | CGI: chr8: | . |
| | | | | | 119832558-119832875 | |
| 338430 | chr14 | CCDC176; | cg18638434 | 74019948 | CGI: chr14: | S_Shore |
| | | ENTPD5 | | | 74018622-74019653 | |
| 343359 | chr1 | NFIA | cg18946602 | 61084310 | CGI: chr1: | S_Shore |
| | | | | | 61083081-61083750 | |
| 382471 | chr4 | CCNG2 | cg21475610 | 77155998 | CGI: chr4: | N_Shore |
| | | | | | 77156842-77158438 | |
| 406409 | chr1 | PDE4B | cg23045908 | 66333736 | CGI: chr1: | . |
| | | | | | 66533045-66533377 | |

Briefly, the beta-values for methylation suggests the percentage of methylated loci at a specific DNA residue. These values can also be reported in m-values. Higher methylation beta value indicates higher preponderance of UM tumor cells with high-risk features and poor outcomes. These biomarkers can be used a set or individually and still provide accurate prognostication. The methylation cutoff (Table 2) is meant to be a binary cutoff to stratify tumors into two major groups (good prognosis vs poor prognosis, low vs high metastatic potential, disomy 3 vs monosomy 3 tumors). Beta values can also have prognostic values irrespective of the cutoff values identified in Table 2. They can help stratifying patients within each group into different groups based on relative methylation values of these biomarkers.

Predicting Chromosome 3 Copy Number Based on Methylation Beta Values

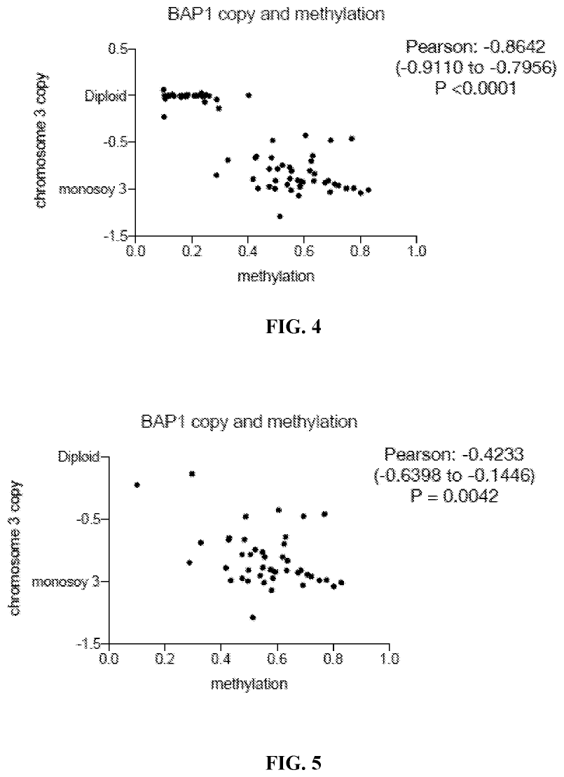

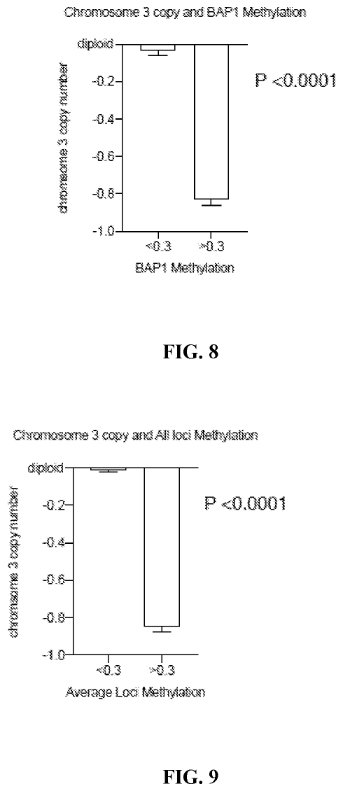

Applicant found that the beta methylation value of BAP1 gene (especially at the locus identified in Table 7) is indicative of genomic copy number of chromosome 3. BAP1 methylation value <0.3 is associated with disomy 3 UM as shown in FIGS. 4 and 8. BAP1 methylation value >0.3 is associated with monosomy 3 UM as shown in FIGS. 4 and 8. Within monosomy 3 UM, higher beta values are associated with more significant copy loss in chromosome 3 or BAP1 gene as shown in FIG. 5. Thereby BAP1 methylation values helped distinguish between disomy 3 tumors, partial monosomy 3 tumors and monosomy 3 tumors.

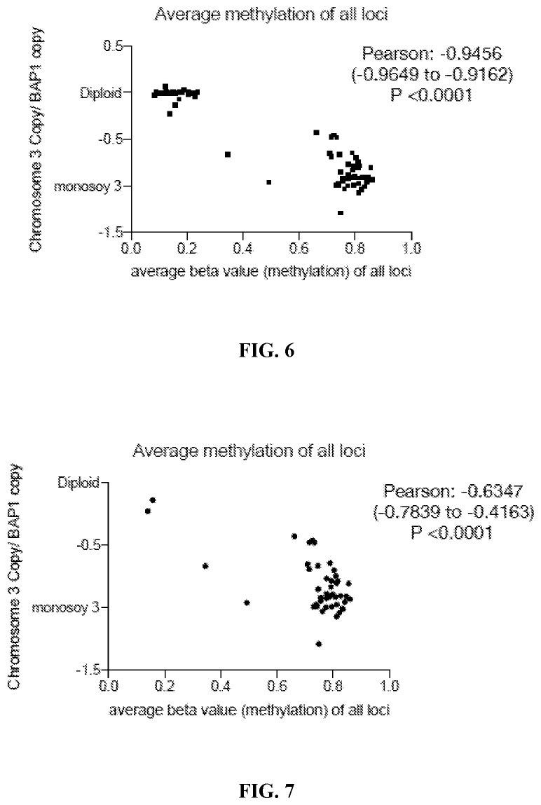

Methylation values of other loci (Tables 2 and 7) also stratified tumors based on methylation beta values of individual loci, average or sum of methylation values of some or all of the genes in Tables 2 and 7. Shown in FIGS. 6, 7 and 9 is chromosom3 copy number as a function of the average methylation of all genes.

Predicting Pathogenic BAP1 Mutations Based on Methylation Beta Values

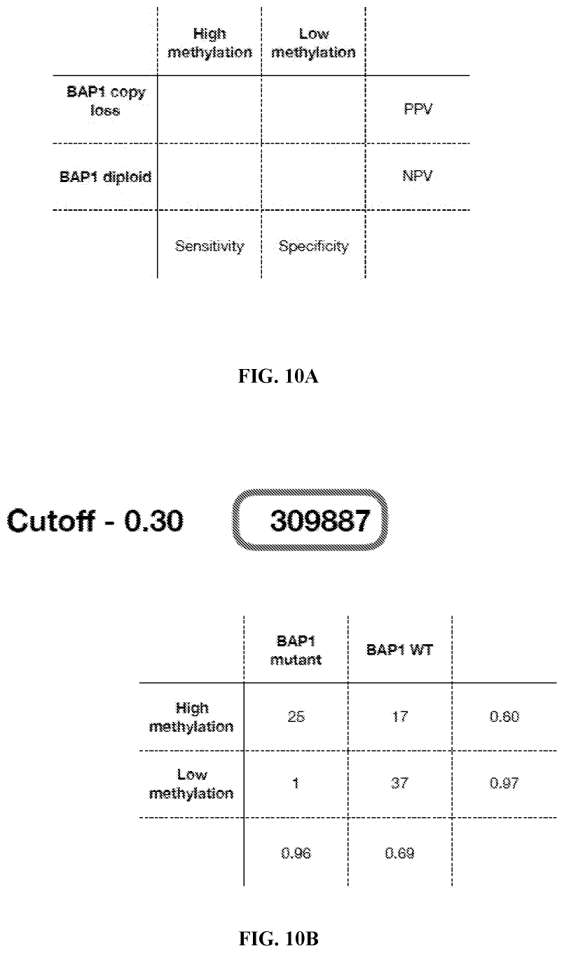

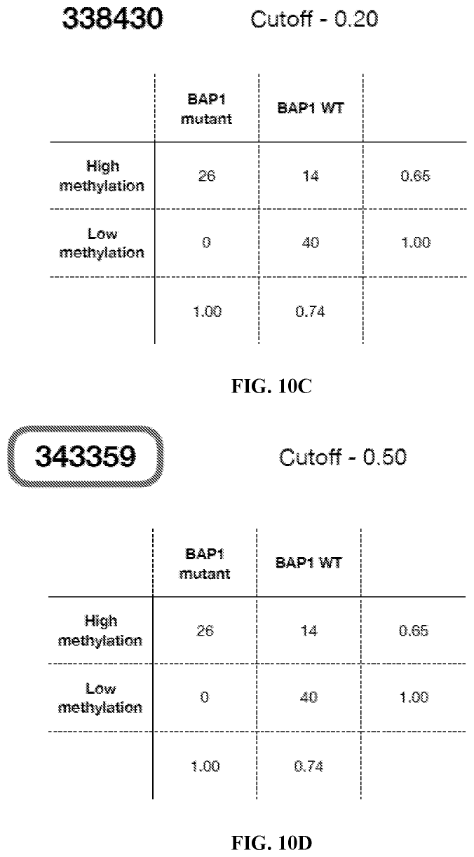

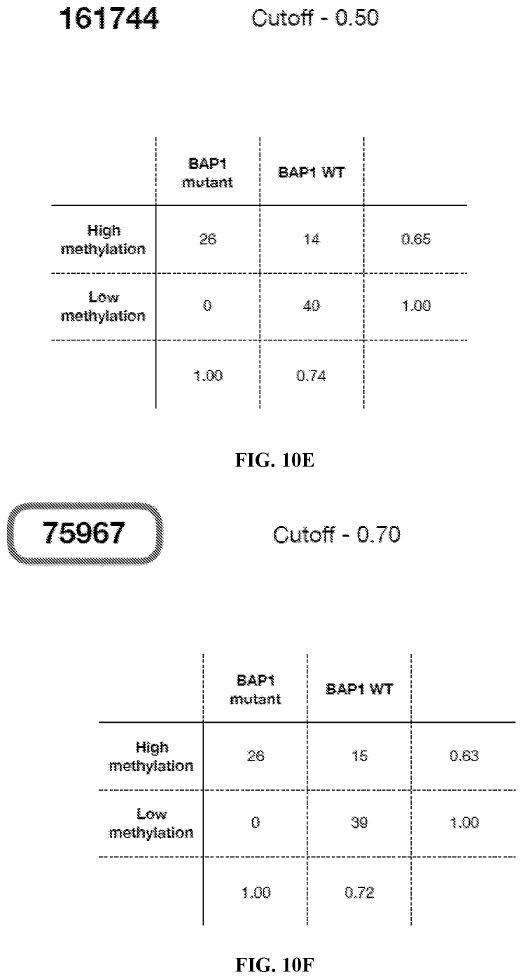

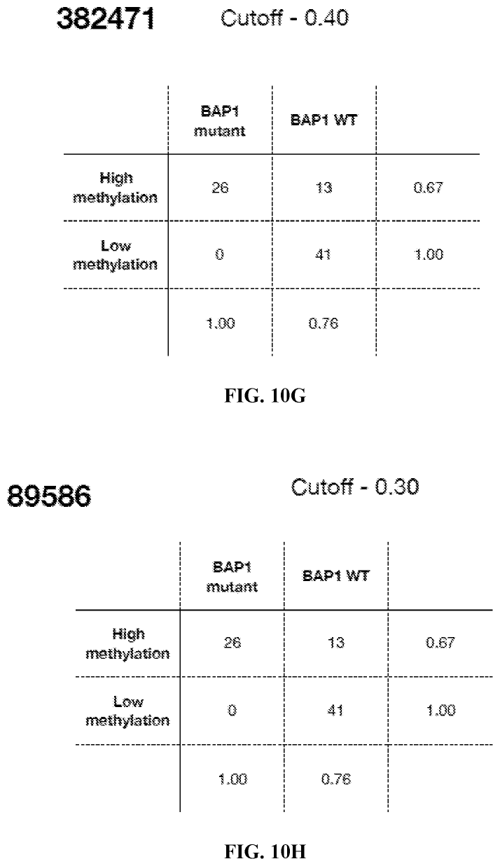

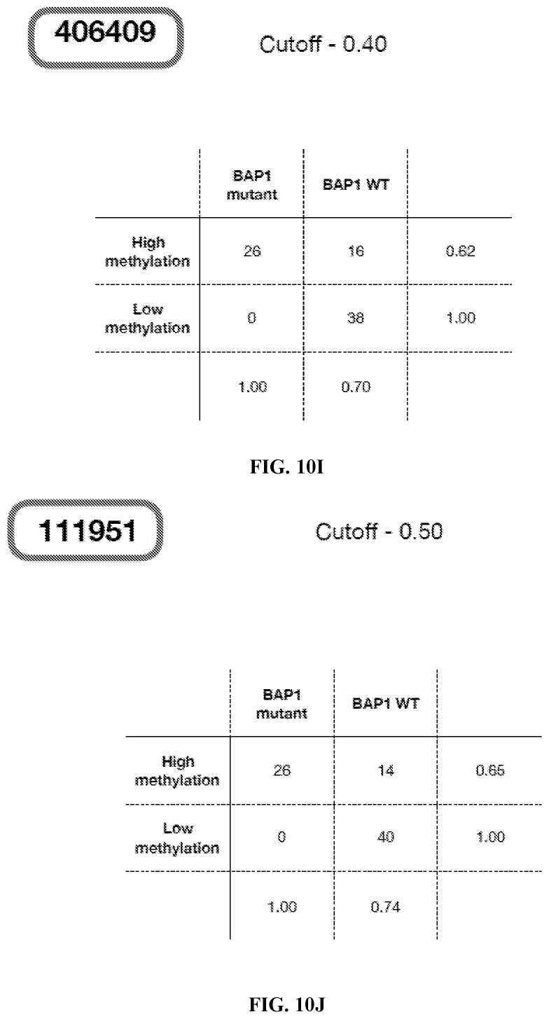

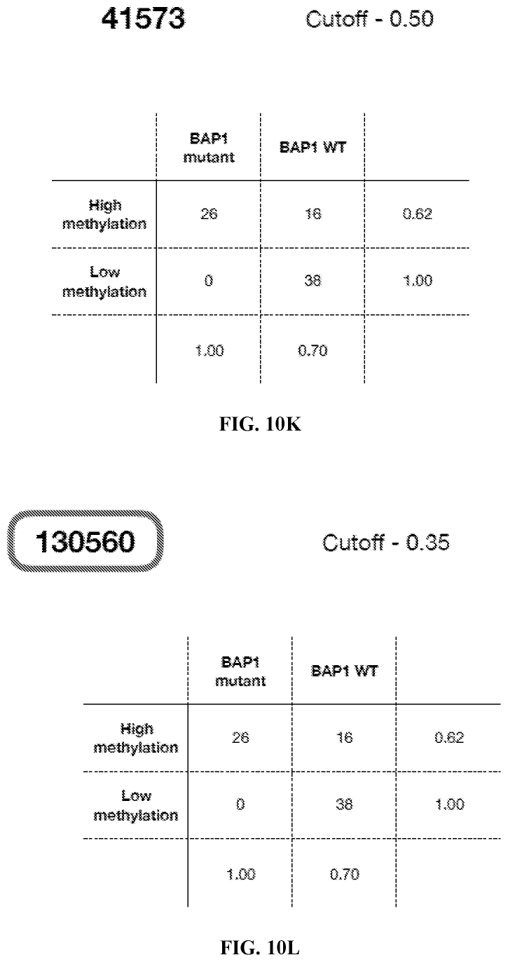

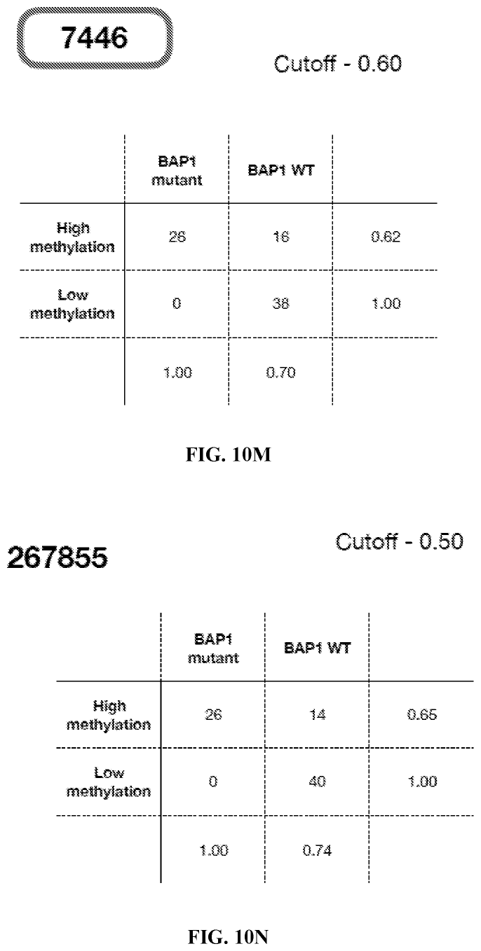

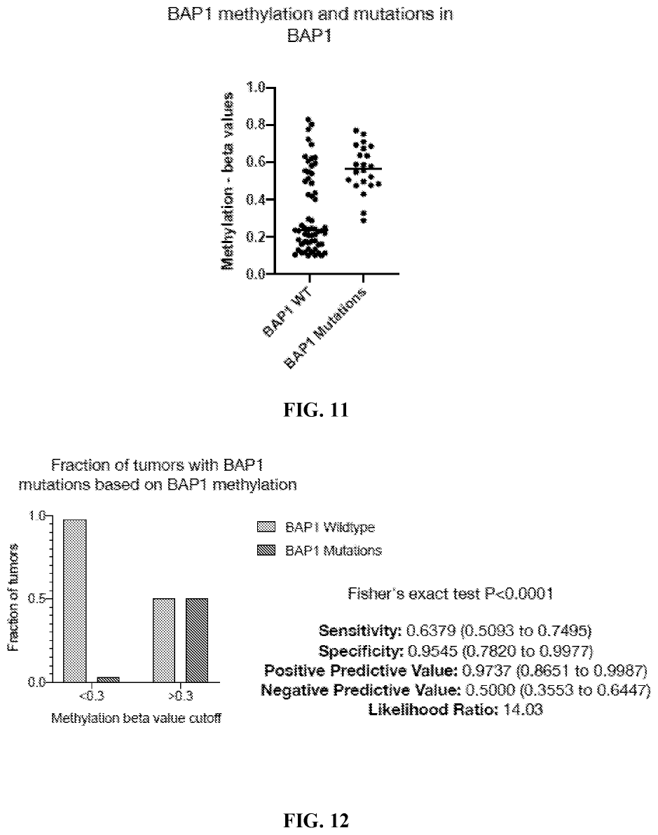

Applicant found that a beta methylation value of BAP1 gene (especially at the locus identified in Table 7) under 0.3 is associated with wildtype BAP1, while a value >0.3 is associated with pathogenic BAP1 mutations (FIGS. 10A-12).

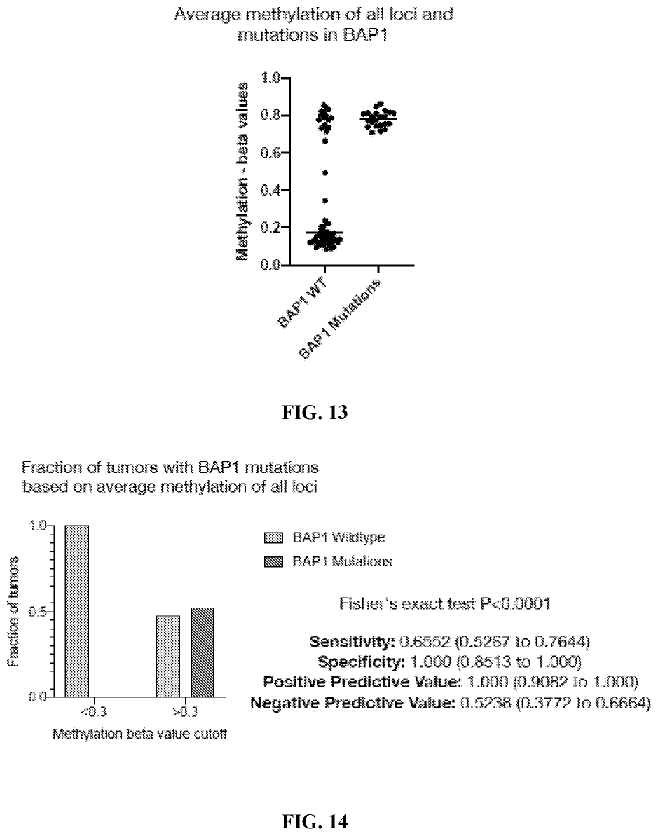

Methylation values of other loci, including or excluding BAP1 (Table 2) also indicated whether the tumor has BAP1 wildtype or pathogenic BAP1 mutations based on methylation beta values of individual loci (Table 2), average or sum of methylation values of some or all of the genes in Table 7, as shown in FIGS. 13-14.

Predicting Overall Survival Based on Methylation Beta Values

Applicant found that a beta methylation value of BAP1 gene (especially at the locus identified in Table 7) under 0.3 is associated with higher overall survival, while a value >0.3 is associated with poor survival (FIG. 17).

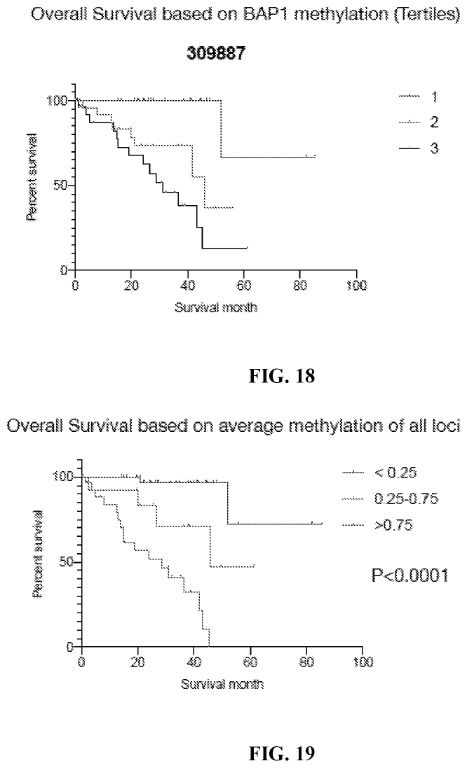

Applicant further found that beta methylation values offer prognostic values beyond a definite cutoff (i.e. 0.3). For example, a beta value at the BAP1 locus (identified in Table 2) of 0.1 confers better prognosis than a beta value of 0.4. A methylation beta value of 0.4 confers a better prognosis than a beta value of 0.8. This is shown in the survival analysis of patients who are stratified based on tertiles of BAP1 methylation beta values (FIG. 18).

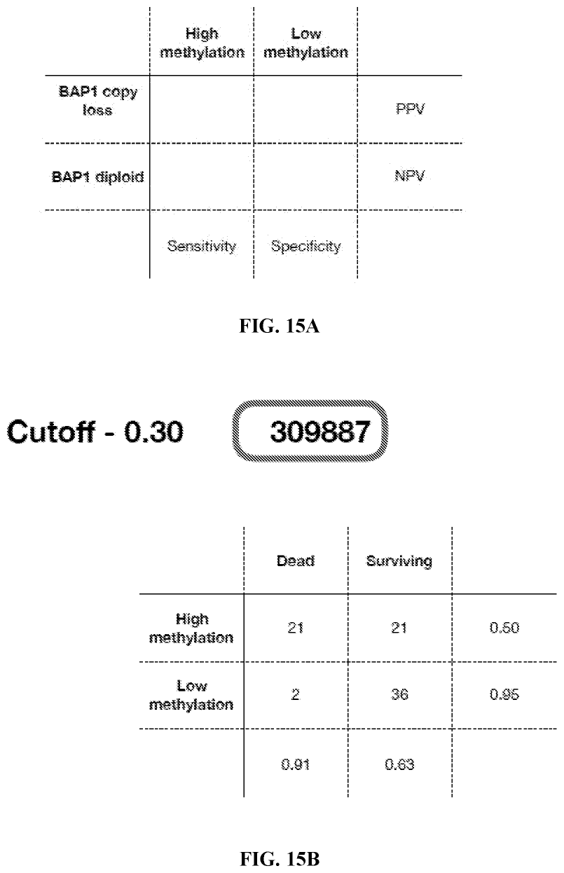

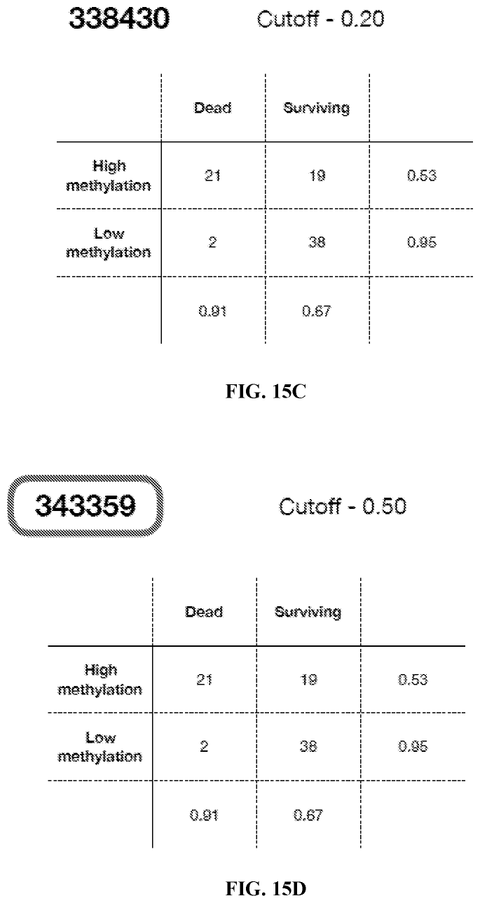

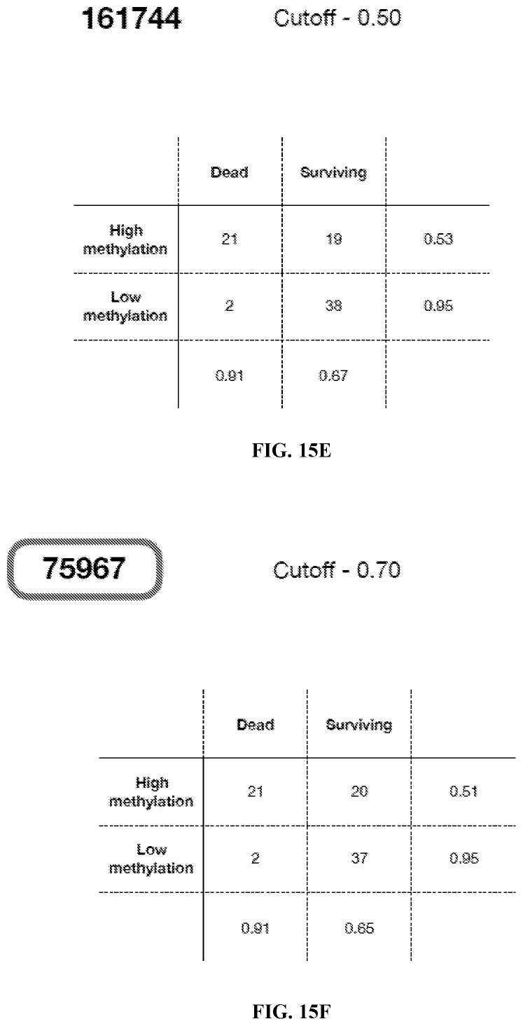

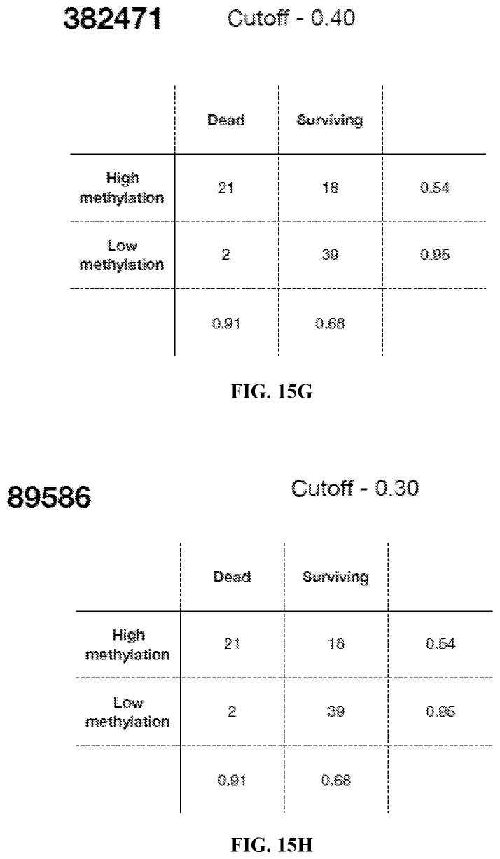

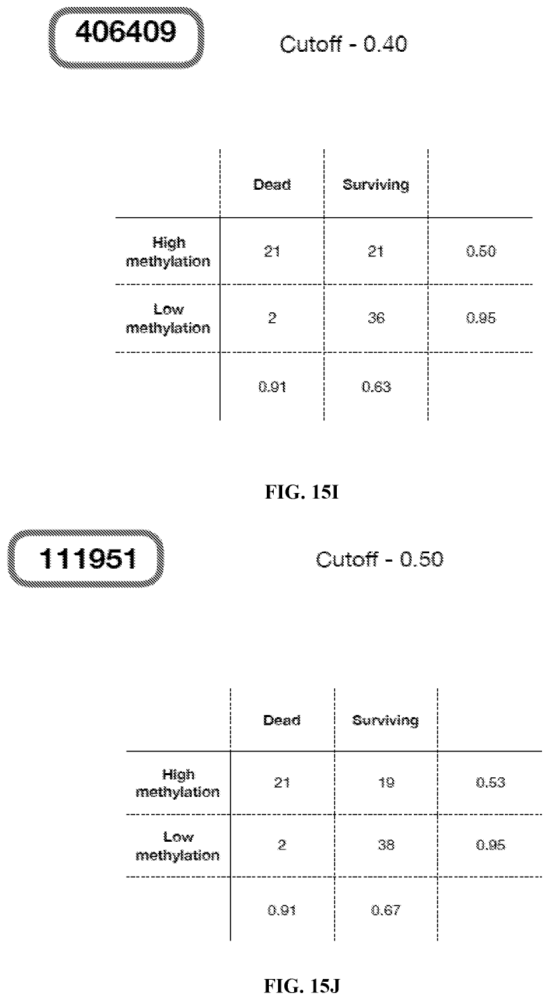

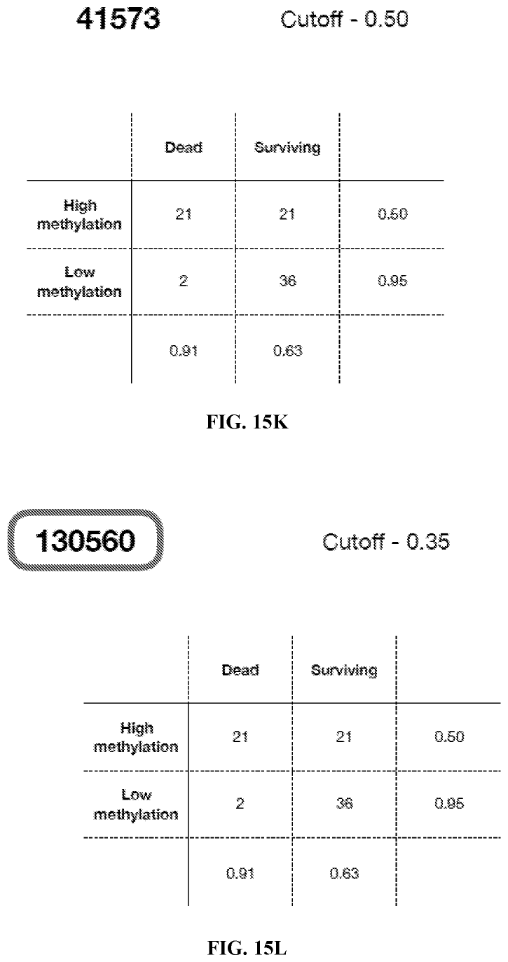

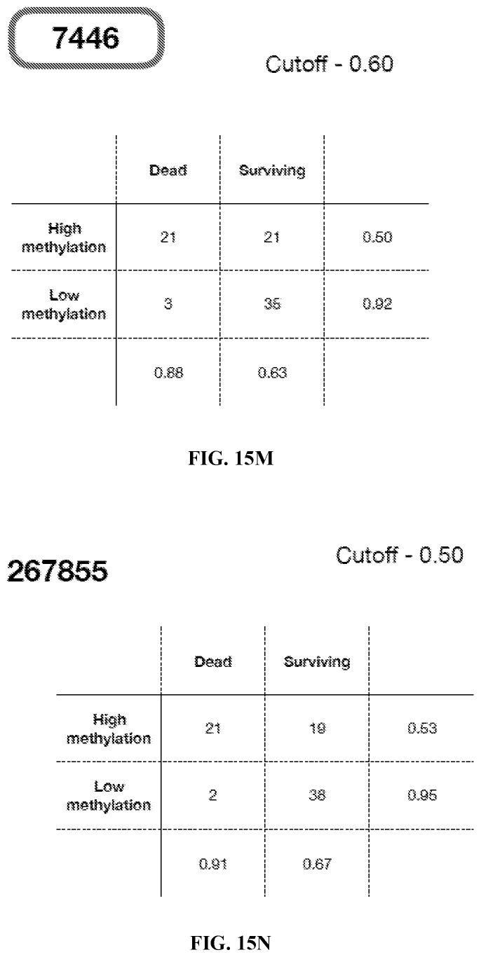

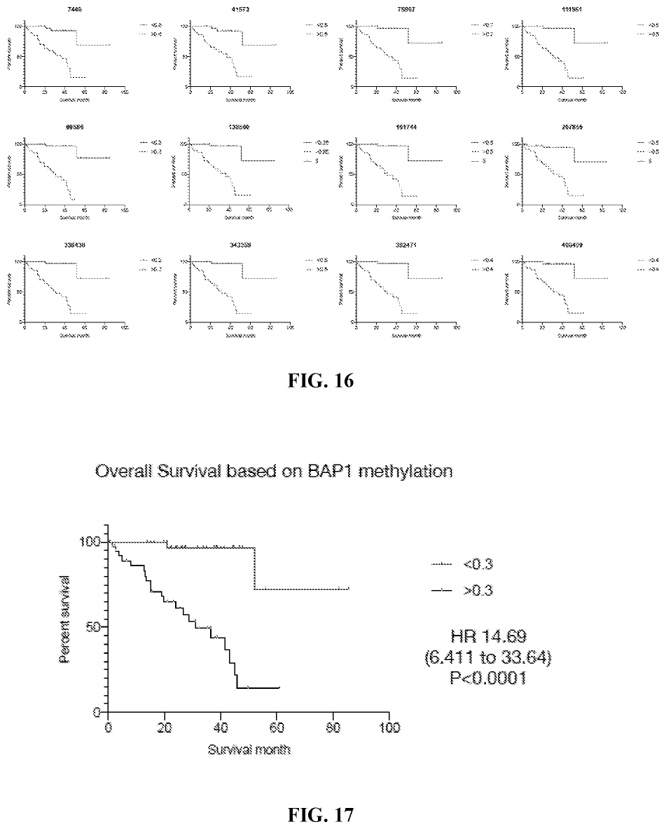

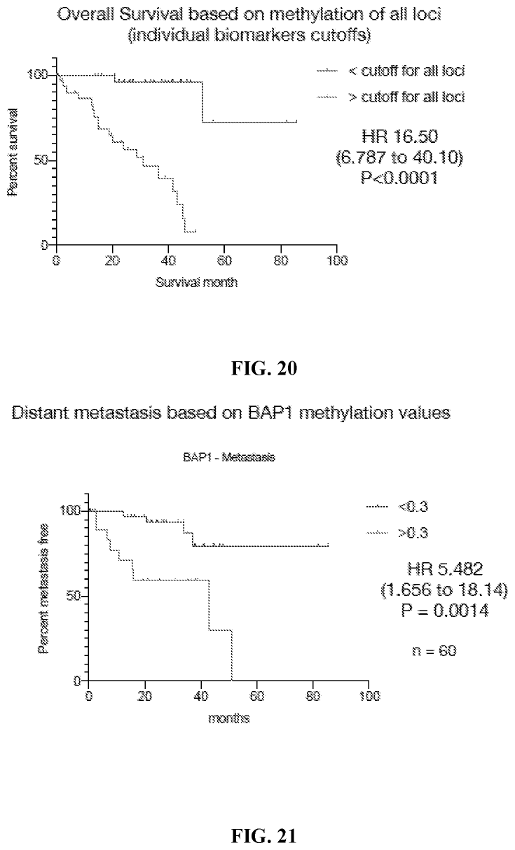

Methylation values of other loci also stratified tumors based on methylation beta values of individual loci, average or sum of methylation values of some or all of the genes in Table 7. Patients were also stratified based on average methylation values for all or some loci to obtain accurate prognostication (<0.25, 0.25-0.75 and >0.75, conferring good, intermediate and poor prognosis) (FIG. 19). Shown in FIG. 19 is the survival analysis of patients based on average methylation beta values of all loci. A positive test was also defined if all loci are above the individual threshold values (Table 2), or below the individual threshold values as demonstrated in survival outcomes shown in FIG. 20. Alternatively, overall survival was estimated based on methylation beta value at each locus (Tables 2 and 7). The beta value cutoffs suggested in Table 2 stratified patients into two major groups with good or poor prognosis based on methylation of individual loci (FIGS. 15A-16H).

Predicting Distant Metastasis to Liver, Lungs and/or Distant Organs Based on Methylation Beta Values

Applicant found that a beta methylation value of BAP1 gene (especially at the locus identified in Table 7) above 0.3 is associated with higher metastatic rates (usually to liver, lungs and/or distant organs), while a value <0.3 is associated with lower metastatic rates (FIG. 21).

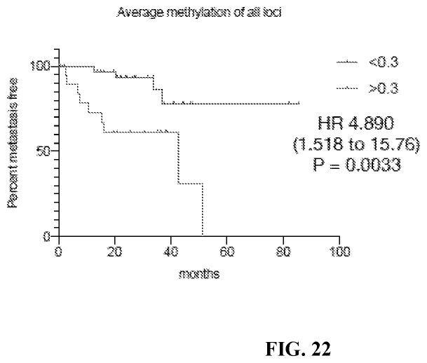

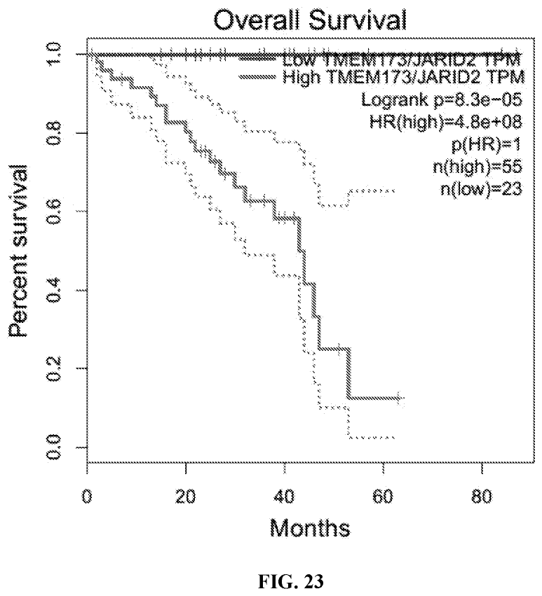

Methylation values of other loci (Tables 2 and 7) also stratified tumors based on methylation beta values of individual loci, average or sum of methylation values of some or all of the genes in Table 7. Patients were also stratified based on average methylation values for all or some loci to obtain accurate prognostication for metastasis. Shown in FIG. 22 is Kaplan Meier curve for metastasis development in patients based on average methylation beta values of all loci.

Similarly Levels of JARID2, TMEM173 (STING) or a Ratio of JARID2 Relative to STING or Vice Versa Helped in Tumor Stratification and Prognostication

Briefly, higher levels of JARID2, lower levels of TMEM173 (STING), higher values of JARID2/TMEM173(STING) and/or lower values of TMEM173(STING)/JARID2 indicated low risk UM with good prognosis; UM tumors with disomy 3; UM tumors with wildtype BAP1, UM tumors with low metastatic tendencies, and/or UM tumors in patients with high overall survival. On the other side, lower levels of JARID2, higher levels of TMEM173 (STING), lower values of JARID2/TMEM173(STING) and/or higher values of TMEM173(STING)/JARID2 indicated high risk UM with poor prognosis; UM tumors with monosomy 3 (partial or full); UM tumors with pathogenic BAP1 mutations; UM tumors with high metastatic tendencies; and/or UM tumors in patients with low overall survival.

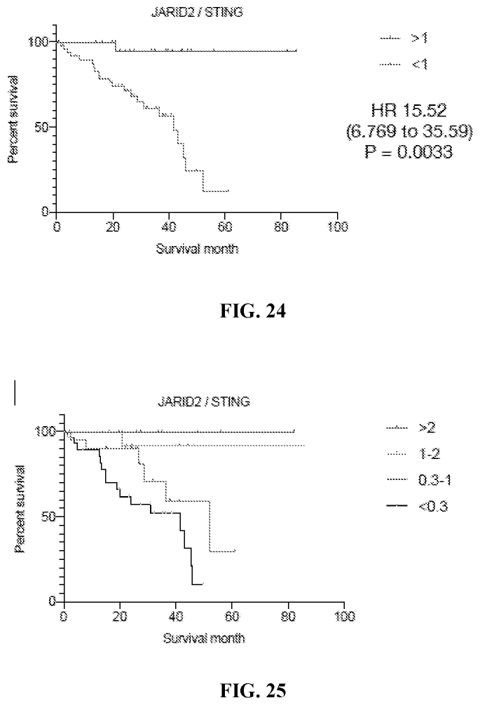

Overall survival of patients was plotted with UM stratified based on JARID2 levels relative to STING (TMEM173) levels. See, FIG. 24. Ratio >1 (upper line) conferred higher chance of survival. Furthermore, FIG. 25 shows stratifying patients based on JARID2/STING(TMEM173) ratios (>2, 1-2, 0.3-1 or less than 0.3) providing better prognostication for overall survival as shown from the Kaplan Meier curves.

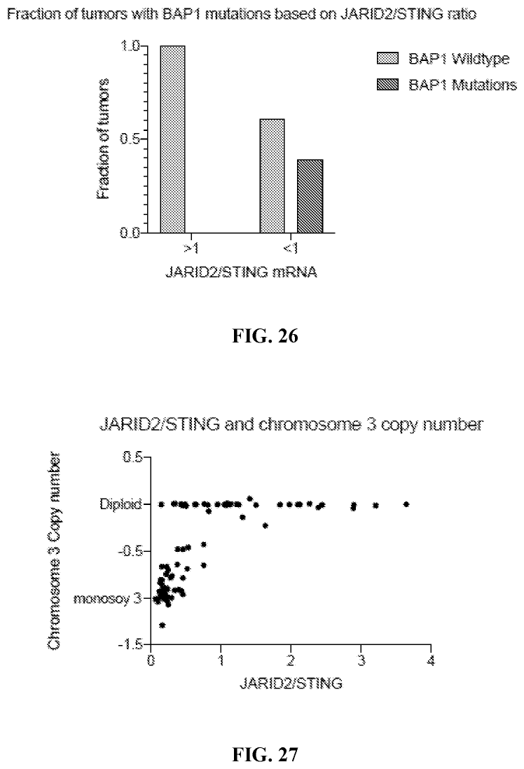

Applicant's analysis further shows that JARID2/STING (TMEM173) ratio >1 was associated with tumors with wildtype BAP1; while JARID2/STING (TMEM173) ratio <1 was associated with tumors with pathogenic BAP1 mutations. See. FIG. 26.

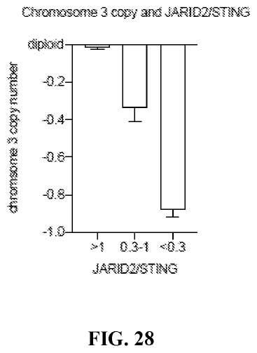

Additionally, JARID2/STING (TMEM173) ratio >1 was associated with tumors with disomy 3, while JARID2/STING (TMEM173) ratio <1 was associated with tumors with monosomy 3. JARID2/STING (TMEM173) ratio of 0.3 to 1 was associated with tumors with monosomy 3 (partial or full). And, JARID2/STING (TMEM173) ratio <0.3 was associated with tumors with full monosomy 3. See. FIGS. 27 and 28.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Additional information regarding the claimed embodiments is provided in the Appendix attached hereto.

Other aspects are set forth within the following claims.

1. A method for treating a high-risk uveal melanoma in a subject having one or more in a sample isolated from the subject:
(a) a high methylation level at one or more of: BAP-1, CCNG2 or FKBP14;
(b) a low mRNA expression ratio of JARID2 to TMEM173;
(c) a high methylation level at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2; or
(d) a high methylation level at one or more of the methylation loci identified in Table 2 and/or 7,
comprising administering to the subject an effective amount of one or more of: an anti-cancer drug, immunotherapy, chemotherapy, and/or radiation therapy selective for the uveal melanoma, thereby treating the subject.

2. The method of claim 1, wherein the low ratio of mRNA expression JARID2 to TMEM173 is less than about 1.

3. The method of claim 1, wherein the low ratio of mRNA expression JARID2 to TMEM173 less than about 2 or less than about 0.3

4. A method for detecting high-risk uveal melanoma in a subject, comprising detecting high methylation levels at one or more of: BAP-1, CCNG2 and/or FKBP14 and/or a low ratio of mRNA expression JARID2 to TMEM173 in a sample isolated from the subject.

5. The method of claim 4, further comprising detecting high methylation levels at one or more of: PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7.

6.-10. (canceled)

11. The method of claim 1, further comprising administering to the subject with a high-risk uveal melanoma an effective amount of one or more of: an anti-cancer drug, immunotherapy, chemotherapy, and/or radiation therapy selective for the uveal melanoma, thereby treating the subject.

12. A method of determining prognosis of a subject having uveal melanoma, comprising detecting methylation levels at one or more of: BAP-1, CCNG2 and/or FKBP14 and/or measuring the mRNA expression of JARID2 and TMEM173 and determining ratio of mRNA expression JARID2 to TMEM173 in a sample isolated from the subject, wherein high methylation levels and/or a low ratio of mRNA expression JARID2 to TMEM173 indicate a less favorable prognosis and wherein low methylation levels and/or a high ratio of mRNA expression JARID2 to TMEM173 indicate a more favorable prognosis.

13.-27. (canceled)

28. A method of treating a subject having uveal melanoma (UM), wherein the subject has one or more of:
(a) low methylation levels at one or more of: BAP-1, CCNG2, FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, ENPP2 and/or methylation loci identified in Table 2 and/or 7 in a sample isolated from the subject; and/or
(b) a high ratio of mRNA expression JARID2 to TMEM173,
in a sample isolated from the subject,
comprising administering an effective amount of one or more of: an anti-cancer drug, immunotherapy, chemotherapy, and/or radiation therapy suitable for the uveal melanoma, thereby treating the subject.

29.-30. (canceled)

31. The method of claim 28, wherein the low methylation levels are detected as a methylation beta value of (a) about 0.3 or lower, or (b) about 0.25 or lower, (c) about 0.75 or lower, or (d) the cutoff for beta value set in Table 2 and/or 7 or lower, and/or wherein the low methylation levels are detected as an average of all methylation beta values at (a) about 0.3 or lower, or (b) about 0.25 or lower, or (c) about 0.75 or lower.

32. (canceled)

33. The method of claim 1, wherein the sample is one or more of a blood, a urine, an aqueous vitreous humor, a tumor biopsy, and/or a liquid biopsy sample.

34. The method of claim 1, wherein the sample is one or more of an aqueous humor, a vitreous humor, a sample from primary or metastatic UM, a tumor tissue in freshly obtained specimens, a tumor tissue in paraffin embedded tissue, a frozen tumor tissue, a tumor tissue in fixative, cell free DAN and/or circulating tumor DNA in biological fluids.

35.-37. (canceled)

38. A kit comprising probes and primers for carrying out the method of claim 1, and optional instructions for use.

39. A method comprising quantifying a methylation level of a loci in a sample isolated from a subject, wherein the loci comprises one or more of the loci identified in Table 2 and/or 7 and/or the loci within one or more of the following genes: BAP-1, CCNG2 and/or FKBP14, PDE4B, SH3D19, SLC6A15 LMCD1, LMCD1-AS1, SENP6, NFIA, CTD-2207O23.12, ZNF358, CCDC176, ENTPD5, DTWD1, FAM227B, SESN1, or ENPP2, wherein optionally, a DNA comprising the one or more loci have been enriched and/or amplified prior to or concurrently with the quantification step.

40.-51. (canceled)

52. A kit comprising probes, primers, optional bisulfate salt or solution, and optional antibodies for carrying out the method of claim 39, and optional instructions for use.
